(19) United States
(12) Patent Application Publication
(10) Pub. No.: US 2024/0350675 A1
(43) Pub. Date: Oct. 24, 2024

(54) CCL20 PROMOTER-REGULATED GENE EXPRESSION

(71) Applicant: Lung Biotechnology PBC, Silver Spring, MD (US)

(72) Inventor: Colin EXLINE, Silver Spring, MD (US)

(21) Appl. No.: 18/611,465

(22) Filed: Mar. 20, 2024

Related U.S. Application Data
(60) Provisional application No. 63/491,485, filed on Mar. 21, 2023.

(51) Int. Cl. A61K 48/00; A61K 38/00; A61P 29/00; A61P 37/06; C07K 14/545; C12N 15/86; G01N 33/68

(52) U.S. Cl. CPC A61K 48/0066; A61P 29/00; A61P 37/06; C07K 14/545; C12N 15/86; G01N 33/6863; A61K 38/00; C12N 2710/16143

(57) ABSTRACT

The present disclosure relates to CCL20 promoters derived from the regulatory region of the CCL20 gene that stimulate gene expression in response to inflammation. These CCL20 promoters, which can be stimulated by an endogenous or exogenous cytokine, can be used to control the timing of the expression of a heterologous gene. The present disclosure also relates to expression cassettes or vectors comprising the CCL20 promoters of the present disclosure operably linked to a nucleic acid sequence encoding, e.g., a polypeptide of interest, as well as delivery systems (e.g., viral particles, lipid vesicles, or nanoparticles) or cells comprising such expression cassettes or vectors. The present disclosure also relates to the use of the promoters, expression cassettes, vectors, delivery systems, or cells in the treatment of diseases related to inflammation, or for recombinant gene expression.

### CROSS-REFERENCE TO RELATED APPLICATIONS AND INCORPORATION BY REFERENCE

This application claims the priority benefit of U.S. Provisional Application No. 63/491,485, filed on Mar. 21, 2023, which is herein incorporated by reference in its entirety. REFERENCE TO SEQUENCE LISTING SUBMITTED ELECTRONICALLY

The content of the electronically submitted ST.26 sequence listing in XML format (Name 5064_0010002_SequenceListing_ST26.xml; Size: 11,037 bytes; and Date of Creation: Mar. 18, 2023) filed with the application is incorporated herein by reference in its entirety.

### FIELD

The present disclosure provides CCL20 promoters and methods related to CCL20 promoter-regulated gene expression in response to inflammation.

### BACKGROUND

Inflammation is a widespread response that is activated by the innate immune system upon detection of signal emanating from damaged tissues or pathogenic microorganisms. Inflammation is generally beneficial for the organism because it alerts the immune system to eliminate the underlying cause and to restore homeostasis. Nevertheless, deregulated and excessive inflammation is harmful and may become chronic due to positive feedback mediated by inflammatory cytokines. The rapid production of pro-inflammatory cytokines, such as tumor necrosis factor alpha (TNF-α), interleukin 1 beta (IL-β), IL-6, and several others, is characteristic of diseases with an inflammatory component.

Inflammation can occur as a response to any harmful stimuli. Physical causes of inflammation include, e.g., burns, frostbite, physical injury, trauma, or ionizing radiation. Biological causes of inflammation include, e.g., infection by pathogens, immune reactions due to hypersensitivity and allergies, or stress. Chemical causes of inflammation include, e.g., chemical irritants or toxins. Inflammatory abnormalities underlie a vast variety of human diseases. The immune system is often involved with inflammatory disorders, as demonstrated in both allergic reactions and some myopathies, with many immune system disorders, e.g., including inflammatory bowel disease (IBD), rheumatoid arthritis (RA), multiple sclerosis, psoriasis, or lupus erythematosus, resulting in abnormal inflammation. Non-immune diseases with causal origins in inflammatory processes include cancer, atherosclerosis, and ischemic heart disease.

Non-steroidal anti-inflammatory drugs (NSAIDs) and corticostesroids are widely used and effective anti-inflammatory therapies, yet they can have adverse side effects. Currently, the most advanced clinically used therapies for inflammatory diseases are neutralizers of TNF-α such as adalimumab, etanercept, and infliximab, and endogenous cytokine antagonists, such as anakinra (an IL-1β antagonist), that specifically and effectively inhibit the activity of pro-inflammatory cytokines.

As inflammation is an essential component of the host defense system, long-term systemic suppression can cause adverse side effects, such as a decreased host immune response against infections and an increased risk of cancer development. Anti-inflammatory therapy that could match the responsiveness and tunability of the natural cellular response, but could be controlled externally, has represented both a challenge and an opportunity for synthetic biology. Synthetic biology allows constructing biological systems that are orthogonal to the existing signaling components yet are able to read the inputs from the cellular processes or external signals and produce the physiologically relevant outputs. Progress has already been made in this direction, such as engineering the bacterium Lactococcus lactis to secrete IL-10, an anti-TNF-α nanobody that has been used to treat murine colitis in situ, or Escherichia coli that can autonomously detect gut inflammation through nitric oxide sensing.

### BRIEF SUMMARY

The present disclosure provides a polynucleotide comprising a promoter region comprising a C—C Motif Chemokine Ligand 20 (CCL20) promoter operatively linked to a heterologous gene, wherein the CCL20 promoter induces expression of the heterologous gene (e.g., the expression of a therapeutic protein or therapeutic nucleic acid) in response to stimulation with a cytokine, e.g., IL-1α. In some aspects, the CCL20 promoter comprises the CCL20 promoter sequence set forth in SEQ ID NO: 1. In some aspects, the CCL20 promoter comprises the CCL20 promoter sequence set forth in SEQ ID NO: 2. In some aspects, the CCL20 promoter comprises the CCL20 promoter sequence set forth in SEQ ID NO: 3. In some aspects, the CCL20 promoter comprises the CCL20 promoter sequence set forth in SEQ ID NO: 4. In some aspects, the CCL20 promoter comprises the CCL20 promoter sequence set forth in SEQ ID NO: 5.

In some aspects, the CCL20 promoter is a functional fragment or a functional variant of the CCL20 promoter sequence set forth in any one of SEQ ID NOS: 1-5. In some aspects, the functional variant is a mutant. In some aspects, the functional fragment has a 5′ truncation, a 3′ truncation, or both relative to a CCL20 promoter sequence set forth in any one of SEQ ID NOS: 1-5. In some aspects, the sequence of the CCL20 promoter has at least about 70%, at least about 75%, at least about 80%, at least about 85%, at least about 90%, at least about 95%, at least about 96%, at least about 97%, at least about 98%, or at least about 99% sequence identity to the CCL20 promoter sequence set forth in any one of SEQ ID NOS:1-5. In some aspects, the sequence of the CCL20 promoter has 1, 2, 3, 4, 5, 6, 7, 8, 9, 10, 11, 12, 13, 14, 15, 16, 17, 18, 19, or 20 point mutations. In some aspects, the point mutations are conservative point mutations.

In some aspects, the heterologous gene encodes a protein. In some aspects, the protein is a therapeutic protein. In some aspects, the therapeutic protein is a chimeric antigenic receptor (CAR), an antibody, a tumor suppressor, an inducer of apoptosis, an enzyme, or a hormone.

The present disclosure also provides a composition comprising (a) a polynucleotide comprising a promoter region comprising a CCL20 promoter of any one of SEQ ID NOS: 1-5 or a functional fragment or functional variant thereof operatively linked to a heterologous gene, wherein the CCL20 promoter induces expression of the heterologous gene in response to stimulation with a cytokine, e.g., IL-1α and (b) a delivery vehicle for delivery of the polynucleotide into a cell, e.g., a cell of a subject, or a cell in vitro. In some aspects, the delivery vehicle is a nucleic acid, a plasmid, a viral vector, a prokaryotic cell, a eukaryotic cell, or a lipid. In some aspects, the lipid is comprised in a lipid vesicle. In some aspects, the lipid vesicle is a micelle, a liposome, a lipid nanoparticle, or an extracellular vesicle. In some aspects, the viral vector is selected from the group consisting of adeno-associated virus, adenovirus, retrovirus, orthomyxovirus, paramyxovirus, papovavirus, picornavirus, lentivirus, herpes simplex virus, vaccinia virus, poxvirus, and alphavirus. In some aspects, the delivery vehicle is inserted into a cell (e.g., a cell of subject, a cell from another donor, or a host cell for cell culture, e.g., for recombinant expression of the heterologous gene) with a construct of the present disclosure, i.e., a construct comprising a CCL20 promoter of any one of SEQ ID NOS: 1-5 or a functional fragment or functional variant thereof operatively linked to a heterologous gene, wherein the CCL20 promoter induces expression of the heterologous gene in response to stimulation with a cytokine, e.g., IL-1α. In some aspects, the delivery vehicle is inserted into a cell via transfection. In some aspects, the transfection method is microinjection, lipofection, electroporation, cationic lipid transfection, microparticles, magnetofection, or any suitable method known in the art. In some aspects, the transfection can be transient. In some aspects, the transfection can be stable. In some aspects, a construct comprising the CCL20 promoter and the heterologous gene can be inserted into the genome of the cell, e.g., using a nuclease such as CRISPR/Cas.

The present disclosure also provides a method of producing a cell that expresses at least one heterologous gene comprising inserting into the cell (e.g., via transfection or microinjection) a composition (e.g., a construct or a vector comprising such construct) comprising a polynucleotide comprising a promoter region, wherein the promoter region comprises a CCL20 promoter of any one of SEQ ID NOS: 1-5 or functional fragment or functional variant thereof operatively linked to the heterologous gene, wherein the CCL20 promoter elicits expression of the heterologous gene (e.g., expression of a therapeutic protein or therapeutic nucleic acid) in response to stimulation with a cytokine, e.g., IL-1α. Also provided is a cell genetically modified to express a heterologous gene, wherein the cell comprises a polynucleotide or a composition disclosed herein. Such cell can be a cell to be administered to a subject (e.g., a cell wherein the heterologous gene encodes an anti-inflammatory cytokine, an antibody, a CAR, etc.). The present disclosure also provides a pharmaceutical composition comprising a polynucleotide, a composition, or a cell disclosed herein and a pharmaceutically acceptable carrier or excipient.

Also provided is a method of inducing, regulating, or enhancing the expression of a heterologous gene (e.g., a heterologous gene encoding an anti-inflammatory cytokine, an antibody, or a CAR), in a subject comprising (a) administering a polynucleotide, a composition, a cell, or a pharmaceutical composition comprising a CCL20 promoter of any one of SEQ ID NOS: 1-5 or functional fragment or functional variant thereof operatively linked to the heterologous gene disclosed herein to the subject; and (b) stimulating the CCL20 promoter with a cytokine, e.g., IL-1α. The present disclosure also provides a method of inducing dose-dependent gene expression of a heterologous gene in a cell comprising (a) contacting the cell with a polynucleotide, a composition, a cell, or a pharmaceutical composition comprising a CCL20 promoter of any one of SEQ ID NOS: 1-5 or functional fragment or functional variant thereof operatively linked to the heterologous gene disclosed herein; and (b) contacting the cell with an effective amount of a cytokine capable of stimulating the CCL20 promoter to induce the gene expression of the heterologous gene in a dose-dependent manner, e.g., IL-1α.

Also provided is a method of controlling or inducing gene expression of a heterologous gene, the method comprising operatively coupling a CCL20 promoter of any one of SEQ ID NOS: 1-5 or functional fragment or functional variant thereof to the heterologous gene, wherein the stimulation of the CCL20 promoter with a cytokine, e.g., IL-1α, controls or induces the expression of the heterologous gene in response to the stimulation.

The present disclosure also provides a method of treating a disease or condition in a subject, or a symptom or sequela of such disease or condition, comprising administering an effective amount of a polynucleotide, a composition, a cell, or a pharmaceutical composition comprising a CCL20 promoter of any one of SEQ ID NOS: 1-5 or functional fragment or functional variant thereof operatively linked to the heterologous gene disclosed herein to the subject. In some aspects, the method further comprises inducing the expression of the therapeutic gene by stimulating the CCL20 promoter with a cytokine, e.g., IL-1α.

Also provided is a method of controlling or reducing inflammation in a subject comprising operatively coupling a CCL20 promoter of any one of SEQ ID NOS: 1-5 or functional fragment or functional variant thereof to a heterologous gene, wherein the stimulation of the CCL20 promoter with a cytokine, e.g., IL-1α, induces the expression of the heterologous gene in response to the stimulation, and wherein the heterologous gene controls or reduces inflammation by increasing or decreasing immune activation, or increasing or decreasing the expression of another gene in an inflammation pathway.

In some aspects of the polynucleotides, compositions, cells, or methods disclosed herein, the cytokine stimulating the CCL20 promoter is selected from the group consisting of IL-1α, IL-1β, TNF (TNF-α or TNF-β), IL6, sIL-6R, IL-17, CD30, or any combination thereof. In some aspects, the cytokine stimulating the CCL20 promoter of the present disclosure (i.e., a CCL20 promoter of any one of SEQ ID NOS: 1-5 or a functional fragment or functional variant thereof) is an interleukin. In some aspects, the interleukin is an interleukin-1. In some aspects, the interleukin is an interleukin-1 alpha. In some aspects, the cytokine stimulating the CCL20 promoter of the present disclosure (e.g., a CCL20 promoter of any one of SEQ ID NOS: 1-5 or a functional fragment or functional variant thereof) is an exogenous cytokine; i.e., it has an external source and it is administered to a subject or added to the culture medium. In some aspects, the cytokine stimulating the CCL20 promoter of the present disclosure (i.e., a CCL20 promoter of any one of SEQ ID NOS: 1-5 or a functional fragment or functional variant thereof) is an endogenous cytokine; i.e., the cytokine is produced by the tissues of the subject. In some aspects, the endogenous cytokine stimulating the CCL20 promoter is the result of inflammation in the subject. In some aspects, the inflammation is naturally occurring. In some aspects, the inflammation is induced. In some aspects, e.g., during recombinant expression, the cytokine stimulating the CCL20 promoter can be added to the medium, or can be secreted by co-cultured cells.

In some aspects of the polynucleotides, compositions, cells, or methods disclosed herein, the use of a CCL20 promoter of the present disclosure increases expression of the heterologous gene by at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 450% or at least about 500% with respect to the expression observed using a reference promoter. In some aspects, the reference promoter is a serum amyloid A3 (SAA3) promoter.

The present disclosure also provides a gene therapy vector comprising a gene (e.g., a CAR) that upon expression increases or decreases immune activation in a subject, wherein the gene is under the control of a CCL20 promoter of the present disclosure (i.e., a CCL20 promoter of any one of SEQ ID NOS: 1-5 or a functional fragment or functional variant thereof). The present disclosure also provides a cell for gene therapy comprising a gene (e.g., a CAR) that upon expression increases or decreases immune activation in a subject, wherein the gene is under the control of a CCL20 promoter of the present disclosure (i.e., a CCL20 promoter of any one of SEQ ID NOS: 1-5 or a functional fragment or functional variant thereof). In some aspects, the cell is an autologous cell, e.g., an autologous T-cell, such as an autologous CAR T-cell. In other aspects, the cell is an allogeneic cell, e.g., an allogeneic T-cell, such as an allogeneic CAR T-cell.

In some aspects, the present disclosure provides a vector comprising a promoter region comprising a CCL20 promoter comprising a sequence set forth in any one of SEQ ID NOS: 1-5 or a functional fragment or functional variant thereof. In some aspects, the vector comprises a heterologous gene operably linked to the CCL20 promoter. In some aspects, the vector does not comprise a heterologous gene operably linked to the CCL20 promoter. In some aspects, the CCL20 promoter is a functional fragment of a sequence set forth in any one of SEQ ID NOS: 1-5. In some aspects, the functional fragment has a 5′ truncation, a 3′ truncation, or both relative to a CCL20 promoter sequence set forth in any one of SEQ ID NOS: 1-5. In some aspects, the CCL20 promoter is a functional variant having a sequence with at least about 70%, about 75%, about 80%, about 85%, about 90%, about 95%, about 96%, about 97%, about 98%, or about 99% sequence identity to the sequence set forth in any one of SEQ ID NOS:1-5. In some aspects, the sequence of the CCL20 promoter functional variant has 1, 2, 3, 4, 5, 6, 7, 8, 9, 10, 11, 12, 13, 14, 15, 16, 17, 18, 19, or 20 point mutations with respect to a sequence set forth in any one of SEQ ID NOS: 1-5. In some aspects, the point mutations are conservative point mutations.

### BRIEF DESCRIPTION OF THE DRAWINGS/FIGURES

FIG. 1A shows the expression levels of firefly luciferase reporter gene upon IL-1α stimulation (fold increase of firefly luciferase) in cells transfected with a Luciferase (FF-Luc) gene (i) in the absence of a promoter, i.e., promoterless-FF-Luc (labeled “Promoterless control”), (ii) under the control of a CCL20 promoter, i.e., CCL20-FF-Luc (labeled “CCL20 promoter”), or (iii) under the control of a SAA3 promoter, i.e., SAA3-FF-Luc (labeled “SAA3 promoter”). Luciferase expression levels were measured after 0.5 hours, 2 hours, 4 hours, and 24 hours of IL-1α stimulation.

FIG. 1B shows normalized expression levels of firefly luciferase reporter gene in the absence of stimulation or after 4 hours of IL-1α stimulation in cells transfected with a Luciferase (FF-Luc) gene (i) in the absence of a promoter, i.e., promoterless-FF-Luc (labeled “Promoterless control”), (ii) under the control of a CCL20 promoter, i.e., CCL20-FF-Luc (labeled “CCL20 promoter”), (iii) under the control of a SAA3 promoter, i.e., SAA3-FF-Luc (labeled “SAA3 promoter”), or (iv) under the control of an SV40 promoter, i.e., SV40-FF-Luc (labeled “SV40 promoter”).

FIG. 2 shows expression levels of Interleukin Receptor Antagonist IL-1RA, an anti-inflammatory cytokine that competes with active IL-1 and blocks binding to their common activating receptor IL-1R1, secreted by cells transfected with (i) Luciferase under the control of a CCL20 promoter (“CCL20-Luc”; negative control), (ii) the IL-1RA gene under the control of a CMV promoter (“CMV-IL1RA”; positive control), and (iii) the IL-1RA under the control of a CCL20 promoter (“CCL20-IL1RA”) in response to stimulation with IL-1α.

FIG. 3A shows expression levels of Interleukin-8 (IL-8), a proinflammatory cytokine with proangiogenic, proliferative, and promotility activities, secreted by cells transfected with (i) Luciferase under the control of a CCL20 promoter (“CCL20-Luc”; negative control), (ii) the IL-1RA gene under the control of a CMV promoter (“CMV-IL1RA”; positive control), and (iii) the IL-1RA under the control of a CCL20 promoter (“CCL20-IL1RA”) in response to stimulation with IL-1αc.

FIG. 3B shows expression levels of Interleukin-6 (IL-6), most commonly considered a proinflammatory cytokine involved in the acute-phase immune response and chronic inflammation, secreted by cells transfected with (i) Luciferase under the control of a CCL20 promoter (“CCL20-Luc”; negative control), (ii) the IL-1RA gene under the control of a CMV promoter (“CMV-IL1RA”; positive control), and (iii) the IL-1RA under the control of a CCL20 promoter (“CCL20-IL1RA”) in response to stimulation with IL-1α.

FIG. 3C shows expression levels of Monocyte Chemoattractant Protein-1 (MCP-1), a chemokine that activates the respiratory burst of monocytes and induces the expression of the pro-inflammatory cytokines IL-6 and IL-10, secreted by cells transfected with (i) Luciferase under the control of a CCL20 promoter (“CCL20-Luc”; negative control), (ii) the IL-1RA gene under the control of a CMV promoter (“CMV-IL1RA”; positive control), and (iii) the IL-1RA under the control of a CCL20 promoter (“CCL20-IL1RA”) in response to stimulation with IL-1α.

FIG. 4 shows the expression level of IL-10 by HeLa cells transfected with a CCL20 promoter-IL10 construct after stimulation with IL-1α.

FIG. 5 shows luciferase expression in a tunable system comprising a Gal4-VP16 synthetic transcription factor under the control of the CCL20 promoter. The expression level of luciferase can be controlled according to the number of upstream activation sequences (UAS) in the luciferase reporter construct.

### DETAILED DESCRIPTION

The present disclosure provides CCL20 promoters that elicit gene expression in response to stimulation with pro-inflammatory cytokines. These promoters, which are sensitive to inflammation, can be used, e.g., to control the timing of the expression of a heterologous gene, e.g., an anti-inflammatory chemokine.

The CCL20 promoters disclosed herein (i.e., a CCL20 promoter of any one of SEQ ID NOS: 1-5 or a functional fragment or variant thereof) can elicit gene expression in response to inflammation, either naturally occurring inflammation or induced inflammation, in a subject. Thus, the CCL20 promoters of the present disclosure can be used to control in a dose-dependent manner the expression level of a gene that, e.g., increases or decreases immune activation, signals to another set of genes to be expressed in an inflammation pathway, or in any way changes or modifies the state of a cell in such a manner that inflammation diminishes. The strength and speed of the gene expression induced by the CCL20 promoters of the present disclosure creates a potent response that can be manipulated for gene therapy, cell therapy, or recombinant expression.

The CCL20 promoters of the present disclosure can be used for example in gene therapy. As a non-limiting example, an individual suffering from an inflammatory condition (e.g., cancer, a neurodegenerative disease, or an autoimmune disease) could receive gene therapy comprising a heterologous gene under the control of a CCL20 promoter of the present disclosure. Endogenously released inflammatory cytokines or exogenous (administered) inflammatory cytokines, e.g., IL-1α, would stimulate the CCL20 promoter and trigger a downstream anti-inflammatory response, e.g., via the expression of an anti-inflammatory cytokine or an anti-inflammatory molecule (e.g., an antibody) encoded by the heterologous gene controlled by the CCL20 promoter. Alternatively, endogenously released inflammatory cytokines or exogenous (administered) inflammatory cytokines may trigger the expression of an intermediate product (e.g., an activator or inhibitor) encoded by the heterologous gene controlled by the CCL20 promoter, which upon action in an inflammatory pathway would result in an anti-inflammatory response.

The CCL20 promoters of the present disclosure (i.e., a CCL20 promoter of any one of SEQ ID NOS: 1-5 or a functional fragment or variant thereof) can also be used to genetically modify cells for administration (e.g., via injection or implantation) to a subject suffering from an inflammatory condition. In some aspects, the cell is an autologous cell, e.g., an autologous T-cell, such as an autologous CAR T-cell. In other aspects, the cell is an allogeneic cell, e.g., an allogeneic T-cell, such as an allogeneic CAR T-cell. In some aspects, the cell can be implanted in the subject.

Stimulation of the CCL20 promoter in the administered/implanted cells can trigger a downstream anti-inflammatory response, e.g., by eliciting the expression of an anti-inflammatory cytokine (e.g., IL1RA, IL-4, IL-10, IL-11, IL-13 protein, TGF-β, leukemia inhibitory factor, interferon-alpha, or a combination thereof if the construct is bicistronic or polycistronic), an analgesic peptide or protein, or a therapeutic protein (e.g., an antibody or another biologic) in response, e.g., to stimulation with an inflammatory cytokine such as IL-1α, IL-1β, TNFα, IL-6, IL-15, IL-17, or IL-18.

In some aspects, the CCL20 promoters of the present disclosure can be indirectly activated by a cytokine such as IL-6, sIL-6R, or IL-17, which indirectly would elicit expression of a heterologous gene under the control of the CCL20 gene via increased recruitment of phosphorylated NF-κB to the CCL20 promoter.

The CCL20 promoters disclosed herein can also be used for recombinant gene expression. In some aspects, recombinant gene expression under the control of a CCL20 promoter disclosed herein can be induced by the addition of an exogenous cytokine to the cell culture medium, e.g., IL-1α. In other aspects, the cultured cell can be subjected to conditions that elicit the production of cytokines by the cultured cell, or by a cell co-cultured with the host cell, which in turn would induce recombinant gene expression under the control of a CCL20 promoter disclosed herein.

Therapeutic uses of the CCL20 promoters of the present disclosure are not limited to the expression of genes triggering an anti-inflammatory response. For example, the CCL20 promoters disclosed herein can be used to trigger the expression of any protein or gene that can treat a disease or condition characterized by elevated levels of inflammatory cytokines. For example, a gene encoding an antimicrobial or antiviral protein, such as an antibody, may be expressed under the control of a CCL20 promoter of the present disclosure in a subject suffering from a microbial or viral infection. Given the dose-dependent gene expression observed upon induction of the CCL20 promoters of the present disclosure, the level of production of such antimicrobial or antiviral proteins may be endogenously controlled by the severity of the inflammatory response to the disease, may be controlled exogenously by administering compounds (e.g., inflammatory cytokines) that would directly activate the CCL20 promoter, or may be controlled by administering compounds that would indirectly elicit the release of inflammatory cytokines which in turn would active the CCL20 promoter.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values that are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

About: The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

Administration: The terms “administration,” “administering,” and grammatical variants thereof refer to introducing a composition comprising a CCL20 promoter disclosed herein (i.e., a CCL20 promoter of any one of SEQ ID NOS: 1-5 or a functional fragment or functional variant thereof), e.g., a polynucleotide, a vector, or a viral particle, into a subject via a pharmaceutically acceptable route. The introduction of a composition into a subject is by any suitable route, including orally, pulmonarily, intranasally, parenterally (intravenously, intra-arterially, intramuscularly, intraperitoneally, or subcutaneously), rectally, intralymphatically, intrathecally, intratumorally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject. In some aspects, a cell is administered. In some aspects, the cell can be implanted.

Antibody: As use herein, the term “antibody” (Ab) shall include, without limitation, a glycoprotein immunoglobulin that binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. In some aspects, the heterologous protein expressed via stimulation of a CCL20 promoter operably linked to a polypeptide encoding the heterologous protein comprises an antibody or antigen-binding portion thereof.

Antigen: The term “antigen” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA.

Antigen binding portion: An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-GD2 antibody, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

CAR: The term “Chimeric Antigen Receptor” or alternatively a “CAR” refers to a set of polypeptides, typically two in the simplest form, which when in an immune effector cell, provides the cell with specificity for a target cell, typically a cancer cell, and with intracellular signal generation. In some aspects, a CAR comprises at least an extracellular antigen-binding domain, a transmembrane domain and a cytoplasmic signaling domain (also referred to as “an intracellular signaling domain”) comprising a functional signaling domain derived from a stimulatory molecule and/or costimulatory molecule as defined below. In some aspects, the heterologous protein expressed via stimulation of a CCL20 promoter operably linked to a polypeptide encoding the heterologous protein comprises a CAR.

CCL20: Chemokine (C—C motif) ligand 20 (CCL20), also known as liver activation regulated chemokine (LARC) or Macrophage Inflammatory Protein-3 (MIP3A) is a small cytokine belonging to the CC chemokine family. It is strongly chemotactic for lymphocytes and weakly attracts neutrophils. CCL20 elicits its effects on its target cells by binding and activating the chemokine receptor CCR6. Gene expression of CCL20 can be induced by microbial factors such as lipopolysaccharide (LPS), and inflammatory cytokines such as tumor necrosis factor and interferon-7, and down regulated by IL-10.

CCL20 promoter: As used herein, the terms “CCL20 promoter,” “CCL20 promoter of the present disclosure,” or “CCL20 promoter disclosed herein” relates to a nucleic acid sequence derived from the regulatory region of the human CCL20 having a promoter activity. The term also encompasses functional fragment and functional variants, i.e., fragments, mutants, and derivatives thereof that have promoter activity. In specific aspects, the term CCL20 promoter refers to a polynucleotide comprising a nucleic acid sequence set for in any one of SEQ ID NOS: 1, 2, 3, 4 or 5, or a functional variant disclosed herein.

Chemokine: The term “chemokine” refers to a family of small cytokines or signaling proteins secreted by cells that induce directional movement of leukocytes, as well as other cell types, including endothelial and epithelial cells. Chemokines have been classified into four main subfamilies: CXC, CC, CX3C and C. All of these proteins exert their biological effects by interacting with G protein-linked transmembrane receptors called chemokine receptors, which are selectively found on the surfaces of their target cells.

Cytokine: The term “cytokine” refers to a broad and loose category of small proteins (˜5-25 kDa) important in cell signaling. Cytokines are peptides and cannot cross the lipid bilayer of cells to enter the cytoplasm. Cytokines have been shown to be involved in autocrine, paracrine and endocrine signaling as immunomodulating agents. Cytokines include chemokines, interferons, interleukins, lymphokines, and tumor necrosis factors, but generally not hormones or growth factors.

Complement: The term “complement” as used herein indicates a sequence that is complementary to a reference sequence. It is well known that complementarity is the base principle of DNA replication and transcription as it is a property shared between two DNA or RNA sequences, such that when they are aligned antiparallel to each other, the nucleotide bases at each position in the sequences will be complementary, much like looking in the mirror and seeing the reverse of things. Therefore, for example, the complement of a sequence of 5′ “ATGC” 3′ can be written as 3′ “TACG” 5′ or 5′ “GCAT” 3′. The terms “reverse complement”, “reverse complementary”, and “reverse complementarity” as used herein are interchangeable with the terms “complement”, “complementary”, and “complementarity.” In some aspects, the term “complementary” refers to 100% match or complementarity (i.e., fully complementary) to a contiguous nucleic acid sequence. In some aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a contiguous nucleic acid sequence.

Complementary: The terms “complementary” and “complementarity” refer to two or more polynucleotides (i.e., each comprising a nucleobase sequence) that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given polynucleotide sequence are matched to the other polynucleotide sequence according to base pairing rules. For example, in some aspects, complementarity between a given polynucleotide sequence and the other polynucleotide sequence may be at least about 70%, at least about 75%, at least about 80%, at least about 85%, at least about 90% or at least about 95%. On the other hand, there may be “complete” or “perfect” (100%) complementarity between a given polynucleotide sequence and the other polynucleotide sequence to continue the example. The degree of complementarity between polynucleotide sequences has significant effects on the efficiency and strength of hybridization between the sequences.

Conserved: As used herein, the term “conserved” refers to nucleotides of a polynucleotide sequence that are occur unaltered in the same position of two or more sequences being compared. Nucleotides that are relatively conserved are those that are conserved amongst more related sequences than nucleotides appearing elsewhere in the sequences.

Corresponding to: The terms “corresponding to” and “corresponds to,” when referencing two separate nucleic acid or nucleotide sequences can be used to clarify regions of the sequences that correspond or are similar to each other based on homology and/or functionality, although the nucleotides of the specific sequences can be numbered differently. In addition, it is recognized that different numbering systems can be employed when characterizing a nucleic acid or nucleotide sequence. Further, it is recognized that the nucleic acid or nucleotide sequences of different variants of a nucleic acid can vary. As used herein, however, the regions of the variants that share nucleic acid or nucleotide sequence homology and/or functionality are deemed to “correspond” to one another.

Culture: The terms “culture”, “cell culture” and “eukaryotic cell culture” as used herein refer to a cell population, either surface-attached or in suspension that is maintained or grown in a medium (see definition of “medium” below) under conditions suitable to survival and/or growth of the cell population. As will be clear to those of ordinary skill in the art, these terms as used herein can refer to the combination comprising the cell population and the medium in which the population is suspended. As used herein, “culturing” refers to growing one or more cells in vitro under defined or controlled conditions. Examples of culturing conditions that can be defined include temperature, gas mixture, time, and medium formulation.

Culture medium: The terms “media”, “medium”, “cell culture medium”, “culture medium”, “tissue culture medium”, “tissue culture media”, and “growth medium” as used herein refer to a solution containing nutrients that can be used to nourish growing cultured host cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors.

Derived from: The terms “derived from” or “derivative” as used herein, refer to a component that is isolated from or made using a specified molecule, or information (e.g., a nucleic acid sequence) from the specified molecule. For example, a polynucleotide sequence that is derived from another polynucleotide sequence can include a polynucleotide sequence that is identical or substantially similar to the polynucleotide sequence it derives from. In the case of polynucleotides, the derived species can be obtained by, for example, naturally occurring mutagenesis, artificial directed mutagenesis, or artificial random mutagenesis. The mutagenesis used to derive polynucleotides can be intentionally directed or intentionally random, or a mixture of each. The mutagenesis of a polynucleotide to create a different polynucleotide derived from the first polynucleotide can be a random event (e.g., caused by polymerase infidelity) and the identification of the derived polynucleotide can be made by appropriate screening methods known in the art. In some aspects, a polynucleotide sequence that is derived from a first polynucleotide sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to the first polynucleotide sequence, respectively, wherein the derived polynucleotide sequence retains the biological activity of the original polynucleotide.

Downstream/Upstream: The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription. The term “upstream” refers to a nucleotide sequence that is located 5′ to a reference nucleotide sequence.

Encoding: The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a nucleotide sequence “encoding” an amino acid sequence,” e.g., a polynucleotide “encoding” a heterologous molecule under the control of a CCL20 promoter of the present disclosure, includes all nucleotide sequences that are degenerate versions of each other and that encode the same heterologous molecule (e.g., the same amino acid sequence).

Expression: The term “expression” as used herein refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide, which is translated from a transcript. In some aspects, the terms “expression” or “expresses” are used to refer to transcription and translation occurring within a cell. The level of expression of a product gene in a host cell can be determined based on either the amount of corresponding mRNA that is present in the cell or the amount of the protein encoded by the product gene that is produced by the cell, or both.

Fragment: As used herein, the term “fragment,” e.g., a fragment of a CCL20 promoter disclosed herein, refers to an polynucleotide sequence of a CCL20-promoter of the present disclosure that is shorter than a CCL20 promoter sequence disclosed herein (e.g., a CCL20 promoter of any one of SEQ ID NOS: 1-5), e.g., 5′-end and/or 3′-end deleted or with any part of the polynucleotide sequence deleted in comparison to the naturally occurring polynucleotide.

Functional fragment/non-functional fragment: As used herein, the term “functional fragment” refers to a polynucleotide fragment derived from a CCL20 promoter sequence disclosed herein that retains promoter function. Accordingly, in some aspects, a functional fragment of a CCL20 promoter disclosed herein, retains the ability to elicit the expression of a heterologous gene that is operably linked to the CCL20 promoter fragment. Conversely, a “non-functional fragment” would lack one or more of the functional characteristics of the parent molecule.

Whether a fragment of a CCL20 promoter disclosed herein is a functional fragment can be assessed by any art known methods without undue experimentation. For example, a reporter gene (e.g., luciferase) can be operably linked to a CCL20 promoter fragment, and the expression of luciferase in response to stimulation with IL-1α can be determined. In some aspects, the functional fragment retains, e.g., at least about 20%, at least about 30%, at least about 40%, at least about 50%, at least about 60%, at least about 70%, at least about 80%, at least about 90% or at least about 100% of the ability of a CCL20 promoter disclosed herein to control the expression of a heterologous gene (as determined, e.g., using a luciferase expression assay in response to stimulation with IL-1α). In some aspects, a CCL20 promoter functional fragment consists of a sequence that is complementary to the reverse complement of a CCL20 promoter of any one SEQ ID NOS: 1-5, and which can hybridize with such reverse complement sequence of a CCL20 promoter of any one of SEQ ID NOS: 1-5 under stringent conditions.

Functional variant/non-functional variant: As used herein, the term “functional variant” refers to a polynucleotide derived from a CCL20 promoter sequence disclosed herein, e.g., via point mutation, insertion, deletion, etc., that retains promoter function. In some aspects, a functional variant of a CCL20 promoter disclosed herein, retains the ability to elicit the expression of a heterologous gene that is operably linked to the CCL20 promoter fragment. Conversely, a “non-functional variant” would lack one or more of the functional characteristics of the parent molecule.

Whether a variant (e.g., a mutant) of a CCL20 promoter disclosed herein is a functional variant can be assessed by any art known methods without undue experimentation. For example, a reporter gene (e.g., luciferase) can be operably linked to a CCL20 promoter variant, and the expression of luciferase in response to stimulation with IL-1α can be determined. In some aspects, the functional variant retains, e.g., at least about 20%, at least about 30%, at least about 40%, at least about 50%, at least about 60%, at least about 70%, at least about 80%, at least about 90% or at least about 100% of the ability of a CCL20 promoter disclosed herein to control the expression of a heterologous gene (as determined, e.g., using a luciferase expression assay in response to stimulation with IL-1α). In some aspects, a CCL20 promoter functional variant consists of a sequence that is complementary to the reverse complement of a CCL20 promoter of any one of SEQ ID NOS: 1-5, and which can hybridize with such reverse complement sequence of a CCL20 promoter of SEQ ID NOS: 1-5 under stringent conditions.

Gene: The terms “gene,” “coding sequence,” “encoding nucleic acid,” and grammatical variants thereof are used interchangeably in the present disclosure and refer to nucleic acids (RNA or DNA molecule) that comprise a nucleotide sequence that encodes a gene of interest, which is generally a protein, e.g., a therapeutic protein such as an antibody or a CAR. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

Heterologous gene: As used herein, the term “heterologous gene” refers to a gene operably linked to a CCL20 promoter of the present disclosure, wherein the gene is not naturally occurring CCL20. In some aspects, the heterologous gene can be a gene encoding a therapeutic protein, e.g., an anti-inflammatory cytokine or an antibody, or a therapeutic nucleic acid, e.g., an RNA interference molecule. In some aspects, in particular for diagnostic use, the heterologous gene encodes a reporter molecule (e.g., luciferase).

Identical: In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “highly conserved” if they are about 70% identical, about 80% identical, about 90% identical, about 95% identical, about 98% identical, or about 99% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Identity: As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and available from the European Bioinformatics Institute (EBI) at www.ebi.ac.uk/Tools/psa. In one particular aspects, sequence identity corresponds to the percentage of sequence identity of a global pairwise alignment determined using a program implementing the Needleman-Wunsch algorithm, e.g., Needle, available at www.ebi.ac.uk/Tools/psa/emboss_needle/.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent of sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

Inflammation: As used herein the term “inflammation” refers to the complex biological response of vascular tissues to harmful stimuli, such as pathogens, damaged cells, or irritants. The classical signs of acute inflammation are pain, heat, redness, swelling, and loss of function. Inflammation is a protective attempt by the organism to remove the injurious stimuli and to initiate the healing process. Inflammation is not a synonym for infection, even though the two are often correlated (the former often being a result of the latter). Inflammation can also occur in the absence of infection, although such types of inflammation are usually maladaptive (such as in atherosclerosis). Inflammation is a stereotyped response, and therefore it is considered as a mechanism of innate immunity, as compared to adaptive immunity, which is specific for each pathogen. Progressive destruction of tissue in the absence of inflammation would compromise the survival of the organism. On the other hand, chronic inflammation might lead to a host of diseases, such as hay fever, periodontitis, atherosclerosis, rheumatoid arthritis, and even cancer (e.g., gallbladder carcinoma). It is for that reason that inflammation is normally closely regulated by the body.

Inflammation can be classified as either acute or chronic. “Acute inflammation” is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as “chronic inflammation”, leads to a progressive shift in the type of cells present at the site of inflammation and is characterized by simultaneous destruction and healing of the tissue from the inflammatory process.

In some aspects, a CCL20 promoter of the present disclosure (i.e., a CCL20 promoter of any one of SEQ ID NOS: 1-5 or a functional fragment or functional variant thereof) can be stimulated by pro-inflammatory cytokines released in response to inflammation, e.g., IL-α. In other aspects, a CCL20 promoter of the present disclosure (i.e., a CCL20 promoter of any one of SEQ ID NOS: 1-5 or a functional fragment or functional variant thereof) can be used to elicit or control the expression of polynucleotides encoding therapeutics agents that can treat, ameliorate, or mitigate the symptoms of cardiac, pulmonary, peripheral, hepatic, kidney, digestive or central nervous system disorders, conditions and diseases which may involve inflammation or an inflammatory process, e.g., heart muscle inflammation (myocarditis), chronic myocarditis, acute myocarditis, viral myocarditis, vasculitis, pancreatitis, peritonitis, rheumatoid diseases, inflammatory disease of the kidney, immunological kidney diseases, kidney transplant rejection, immune complex-induced kidney disease, nephropathy induced by toxins, contrast medium-induced nephropathy, diabetic and non-diabetic nephropathy, pyelonephritis, renal cysts, nephrosclerosis, hypertensive nephrosclerosis, nephrotic syndrome, chronic interstitial inflammations, inflammatory bowel diseases (IBD), Crohn's disease, ulcerative colitis (UC), inflammatory skin diseases, inflammatory diseases of the eye, blepharitis, dry eye syndrome, Sjögren's Syndrome, or eye fibrosis. Additional indications that can be treated using the compositions and methods disclosed are disclosed more in detail below.

As used herein, the term “naturally occurring inflammation” refers to an inflammatory response that is the result of the natural course of a disease or condition, or other causes such as exposure to allergens or irritants. In other words, a naturally occurring inflammation is not the result of an external intervention to stimulate a CCL20 promoter of the present disclosure, e.g., the injection of an exogenous interleukin. As used herein, the term “induced inflammation” refers to an external intervention, e.g., increase in temperature, exposure to an antigen or allergen, injection of an exogenous interleukin, etc. to trigger an inflammatory response with the purpose of stimulating a CCL20 promoter of the present disclosure.

Inflammatory cytokine: The terms “inflammatory cytokine” and “proinflammatory cytokine” are used interchangeably and refer to a type of signaling molecule (a cytokine) that is secreted from immune cells like helper T cells (Th) and macrophages, and certain other cell types that promote inflammation. They include, e.g., interleukin-1 (IL-1), IL-6, IL-12, and IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions. Excessive chronic production of inflammatory cytokines contribute to inflammatory diseases that have been linked to different diseases, such as atherosclerosis and cancer.

As used herein, the term “exogenous cytokine” refers to a cytokine that has an external source and that is administered to a subject or cell or added to a culture medium. In some aspects, the exogenous cytokine is an exogenous interleukin (e.g., an interleukin injected to a subject or added to a culture medium). As used herein, the term “endogenous cytokine” refers to a cytokine that is produced by the tissues or cells of a subject or naturally produced by cells in a culture. In some aspects, the endogenous cytokine is an endogenous interleukin, e.g., an interleukin released by the tissues of a subject because of naturally occurring or induced inflammation.

Immune modulator: As used herein, the term “immune modulator” refers to an agent that regulates the immune system. Non-limiting examples of genes encoding immune modulators that can be under the control of a CCL20 promoter of the present disclosure include genes encoding agents such as, modulators of checkpoint inhibitors, ligands of checkpoint inhibitors, cytokines, derivatives thereof, or any combination thereof. In some aspects of the present disclosure, the heterologous gene under the control of a CCL20 promoter of the present disclosure is an immune modulator. The immune modulator gene under the control of a CCL20 promoter of the present disclosure (i.e., a CCL20 promoter of any one of SEQ ID NOS: 1-5 or a functional fragment or functional variant thereof) can also include, for example, a polynucleotide encoding an agonist protein, an antagonist protein, an antibody, an antigen-binding fragment, or a polynucleotide such as siRNA, miRNA, lncRNA, mRNA or DNA.

Immune response: Inflammation is the immune system's response to harmful stimuli, such as pathogens, damaged cells, toxic compounds, or irradiation. An “immune response”, as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses.

In some aspects, the compositions and methods of a CCL20 promoter of the present disclosure (i.e., a CCL20 promoter of any one of SEQ ID NOS: 1-5 or a functional fragment or functional variant thereof) can eliciting an immune response, which is generally an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A stimulatory immune response is an immune response that results in the generation of effectors cells (e.g., cytotoxic T lymphocytes) that can destroy and clear a target antigen (e.g., tumor antigen or viruses).

Interleukin: The term “interleukin”, abbreviated IL, refers to a group of cytokines (secreted proteins and signal molecules) that are expressed and secreted by white blood cells (leukocytes) as well as some other body cells. The human genome encodes more than 50 interleukins and related proteins. As used herein, the term encompasses, e.g., interleukin 1 (interleukin 1 alpha, IL-1α, and interleukin 1 beta, IL-1β), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8; CXCL8), interleukin 9 (IL-9), interleukin 10 (IL-10), interleukin 11 (IL-11), interleukin 12 (IL-12), interleukin 13 (IL-13), interleukin 14 (IL-14), interleukin 15 (IL-15), interleukin 16 (IL-16), interleukin 17 (IL-17), interleukin 18 (IL-18), interleukin 19 (IL-19), interleukin 20 (IL-20), interleukin 21 (IL-21), interleukin 22 (IL-22), interleukin 23 (IL-23), interleukin 24 (IL-24), interleukin 25 (IL-25), interleukin 26 (IL-26), interleukin 27 (IL-27), interleukin 28 (IL-28), interleukin 29 (IL-29), interleukin 30 (IL-30), interleukin 31 (IL-31), interleukin 32 (IL-32), interleukin 33 (IL-33), interleukin 35 (IL-35), and interleukin 36 (IL-36),

Mismatch: The terms “mismatch” or “mismatches” refer to one or more nucleobases (whether contiguous or separate) in an oligomer nucleobase sequence that are not matched to a target pre-mRNA according to base pairing rules. While perfect complementarity is often desired, some aspects can include one or more but preferably 6, 5, 4, 3, 2, or 1 mismatches with respect to the target pre-mRNA. Variations at any location within the oligomer are included. In certain aspects, antisense oligomers of the disclosure include variations in nucleobase sequence near the termini, variations in the interior, and if present are typically within about 6, 5, 4, 3, 2, or 1 subunits of the 5′ and/or 3′ terminus. In certain aspects, one, two, or three nucleobases can be removed and still provide on-target binding.

Nucleic acid: The terms “Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA, and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

Nucleic acid sequence: The terms “nucleic acid sequence” and “nucleotide sequence” are used interchangeably and refer to a contiguous nucleic acid sequence. The sequence can be either single stranded or double stranded DNA or RNA, e.g., a gRNA.

Operably linked: “Operably linked” refers to a juxtaposition wherein the components so described are in a relationship permitting them to function in their intended manner. For instance, a promoter is operably linked to a coding sequence if the promoter affects its transcription or expression. For example, a CCL20 promoter of the present disclosure (i.e., a CCL20 promoter of any one of SEQ ID NOS: 1-5 or a functional fragment or functional variant thereof) can be operably linker to a polynucleotide encoding, e.g., an anti-inflammatory protein, an antibody, a CAR, etc.

Pharmaceutically-acceptable: The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. The term includes excipients and carriers that are useful in preparing a pharmaceutical composition and are generally safe, non-toxic, and desirable.

Pharmaceutical composition: As used herein, the term “pharmaceutical composition” refers to one or more of the compounds mixed or intermingled with, or suspended in one or more other chemical components, such as pharmaceutically acceptable carriers and excipients. One purpose of a pharmaceutical composition is to facilitate administration of preparations of a medicament to a subject.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”).

More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure a polynucleotide can be, e.g., an RNA, e.g., mRNA, or DNA.

Polypeptide: The terms “polypeptide” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly, disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, about 10, about 15, about 20, about 25, about 30, about 35, about 40, about 45, or about 50 amino acids long.

Prevent: The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment. As used herein, “prophylactic” refers to a therapeutic or course of action used to prevent the onset of a disease or condition, or to prevent or delay a symptom associated with a disease or condition. As used herein, a “prophylaxis” refers to a measure taken to maintain health and prevent or delay the onset of a disease or condition, or to prevent or delay symptoms associated with a disease or condition.

Recombinant: A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides that have been separated, fractionated, or partially or substantially purified by any suitable technique.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

Subject: The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications. As used herein, the phrase “subject in need thereof” includes subjects, such as mammalian subjects, that would benefit from administration of a therapeutic agent, e.g., a gene encoding a therapeutic protein wherein the gene is under the control of a CCL20 promoter of the present disclosure (i.e., a CCL20 promoter of any one of SEQ ID NOS: 1-5 or a functional variant thereof).

Subsequence: As used herein, the term “subsequence” refers to a subset of contiguous nucleotides or amino acids in a sequence (either the physical sequence or its symbolic representation).

Therapeutically effective amount: As used herein the term “therapeutically effective amount” is the amount of reagent or pharmaceutical compound, e.g., a vector comprising a gene encoding a therapeutic protein wherein the gene is under the control of a CCL20 promoter of the present disclosure (i.e., a CCL20 promoter of any one of SEQ ID NOS: 1-5 or a functional variant thereof), that is sufficient to a produce a desired therapeutic effect, pharmacologic and/or physiologic effect on a subject in need thereof. A therapeutically effective amount can be a “prophylactically effective amount” as prophylaxis can be considered therapy.

Treating: The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen, or reducing inflammation.

Modulate: As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

Vector: The terms “vector,” “expression vector,” “plasmid,” and grammatical variants thereof are used interchangeably in the present disclosure and refer to polynucleotide exogenous to the genome of a host cell, which is inserted into a particular location in the genome of a host cell (e.g., a T cell). In general, the plasmid comprises a plurality of elements such a recombination sites (e.g., homologous recombination sites and/or site-specific recombination sites), markers (e.g., detection markers and/or selection markers), one or more expression cassettes, or any combination thereof. In some aspects, the plasmid can be a linear plasmid. In other aspects, the plasmid can be a circular plasmid, e.g., an intact circular plasmid.

### II. CCL20 Cytokine-Inducible Promoters and their Uses

The present disclosure provides promoters derived from the regulatory region of human C—C Motif Chemokine Ligand 20 (CCL20) and their uses, e.g., for in situ protein or recombinant gene expression via controlling of the CCL20 promoter activity by a cytokine. The CCL20 promoters disclosed herein can be stimulated by cytokines, e.g., pro-inflammatory cytokines such as IL-1α, to induce the expression of a heterologous gene operably linked to the CCL20 promoter, for example, a gene encoding a heterologous protein, e.g., an anti-inflammatory cytokine. As used herein, the terms “heterologous gene” and “heterologous protein” refer, respectively, to any gene that does not encode CCL20, or a protein that is not CCL20. In some aspects, the expression product of a heterologous gene is a therapeutic protein. In other aspects, the expression product of a heterologous gene is a therapeutic polypeptide.

The CCL20 promoters of the present disclosure (i.e., a CCL20 promoter of any one of SEQ ID NOS:1-5 or a functional fragment or functional variant thereof) can be used, for example, to induce the expression of a heterologous gene operably liked to the CCL20 promoter which encodes, e.g., a heterologous protein or more than one heterologous proteins (e.g., of controlling the expression of genes that are part of a bi- or multi-cistronic construct) in a subject in response to stimulation of the CCL20 promoter by inflammatory cytokines, e.g., IL-α. The inflammatory cytokines capable of inducing heterologous gene expression via stimulation of a CCL20 promoter of the present disclosure can be endogenous (i.e., the result of naturally occurring inflammation) or exogenous (i.e., cytokines administered to a subject to elicit an inflammatory response, or added to a culture medium in the case of recombinant expression).

In some aspects, CCL20 promoters of the present disclosure (i.e., a CCL20 promoter of any one of SEQ ID NOS:1-5 or a functional fragment or functional variant thereof) can be used to control a heterologous gene in subject, wherein the process does not comprise inserting the heterologous gene in the subject. For example, a CCL20 promoter of the present disclosure can be inserted at a locus upstream from a heterologous gene in the genome of a subject, wherein the CCL20 promoter replaces the naturally occurring promoter, e.g., using gene editing methods. In some aspects, the substitute CCL20 promoter can be inserted at a location that inactivates the naturally occurring promoter, e.g, an internal location, without removing the naturally occurring promoter. In some aspects, the substitute CCL20 promoter can be inserted at a location that removes a portion of the naturally occurring promoter, e.g, part of the 3′ terminal region of the naturally occurring promoter, and inactivates the naturally occurring promoter. Accordingly, if, for example, the expression level of a particular gene is too low or it would be beneficial to be under an external control such as the administration of an exogenous cytokine, the naturally occurring promoter may be replaced with a CCL20 promoter of the present disclosure (i.e., a CCL20 promoter of any one of SEQ ID NOS:1-5 or a functional fragment or functional variant thereof).

In some aspects, the heterologous protein or proteins expressed in response to the stimulation of the CCL20 promoter with an endogenous or exogenous cytokine, e.g., IL-α, can be an anti-inflammatory protein, e.g., an antibody or antigen binding portion thereof, a receptor, an antagonist, or any combination thereof. In some aspects, the antibody or antigen binding portion thereof is an anti-TNF antibody, e.g., an antibody against TNF-α. In other aspects, the antibody or antigen binding portion thereof is an anti-IL-1 antibody, e.g., an antibody against IL-1α. In some aspects, the receptor is a soluble TNF receptor. In some aspects, the receptor is a soluble IL-1 receptor. In some aspects, the antagonist in a TNF receptor antagonist. In some aspects, the antagonist can be an IL-1 receptor antagonist. In general, the stimulatory activity of the CCL20 promoter can be elicited by any action that increases the level of a protein that binds to the CCL20 promoter, e.g., NF-kB, STAT3, AP-1, AP-2, C-EBP, SP1, or ESE-1. Exemplary pathways that can result in a downstream increase in levels of NF-kB, STAT3, AP-1, AP-2, C-EBP, SP1, or ESE-1, are depicted, for example, in the signaling pathway diagrams in www.cellsignal.com/pathways/by-research/immunology-inflammation-pathways, all of which are herein incorporate by reference in their entireties.

In some aspects, the heterologous protein or proteins expressed in response to the stimulation of the CCL20 promoter of the present disclosure (i.e., a CCL20 promoter of any one of SEQ ID NOS:1-5 or a functional fragment or functional variant thereof) with an endogenous or exogenous cytokine can be a protein activator or inhibitor that, by modifying the response of an inflammatory pathway, results in a decreased inflammatory response. Three common transcription factors serve as key modulators in the inflammatory response pathway—nuclear factor kappa-light-chain-enhancer of activated B cells (NF-κB), hypoxia-inducible factors-1 alpha (HIF-1α) and signal transducer and activator of transcription (STAT). When inflammation becomes chronic these factors can lead to cancer. See, e.g., Bharat B. et al (2009) Clin Cancer Res 15(2): 425-430; Chiara P. et al (2009) Immunobiology 214(2009): 761-777. Thus, in some aspects, the heterologous protein or proteins expressed in response to the stimulation of the CCL20 promoter of the present disclosure (i.e., a CCL20 promoter of any one of SEQ ID NOS:1-5 or a functional fragment or functional variant thereof) is an antagonist (e.g., an antibody, antigen binding portion thereof, or molecule derived from such antibody, e.g., a CAR) that targets NF-κB, HIF-1α, or STAT. In other words, stimulation of the CCL20 promoter by inflammation would trigger a negative feedback look that would reduce inflammation.

In some aspects, the CCL20 promoters of the present disclosure (i.e., a CCL20 promoter of any one of SEQ ID NOS:1-5 or a functional fragment or functional variant thereof) can be used to induce the expression of a protein in vivo (e.g., after gene therapy), ex vivo, or in vitro (e.g., recombinant expression in cell culture).

In some aspects, the present disclosure provides a polynucleotide comprising a promoter region comprising a CCL20 promoter of the present disclosure (i.e., a CCL20 promoter of any one of SEQ ID NOS:1-5 or a functional fragment or functional variant thereof) operatively linked to a heterologous gene, wherein the CCL20 promoter induces expression of the heterologous gene in response to stimulation with a cytokine, e.g., IL-α. In some aspects, the cytokine used to stimulate a CCL20 promoter of the present disclosure is a pro-inflammatory cytokine.

In some aspects, the CCL20 promoter of the present disclosure (i.e., a CCL20 promoter of any one of SEQ ID NOS:1-5 or a functional fragment or functional variant thereof) is stimulated with interleukin-1 (IL-1), e.g., IL-1α or IL-1β. In some aspects, the CCL20 promoter of the present disclosure (i.e., a CCL20 promoter of any one of SEQ ID NOS:1-5 or a functional fragment or functional variant thereof) is stimulated with interleukin 6 (IL-6). In some aspects, the CCL20 promoter of the present disclosure (i.e., a CCL20 promoter of any one of SEQ ID NOS:1-5 or a functional fragment or functional variant thereof) is stimulated with interleukin 12 (IL-12). In some aspects, the CCL20 promoter of the present disclosure (i.e., a CCL20 promoter of any one of SEQ ID NOS:1-5 or a functional fragment or functional variant thereof) is stimulated with interleukin 18 (IL-18). In some aspects, the CCL20 promoter of the present disclosure (i.e., a CCL20 promoter of any one of SEQ ID NOS:1-5 or a functional fragment or functional variant thereof) is stimulated with tumor necrosis factor alpha (TNF-α). In some aspects, the CCL20 promoter of the present disclosure (i.e., a CCL20 promoter of any one of SEQ ID NOS:1-5 or a functional fragment or functional variant thereof) is stimulated with interferon gamma (IFNγ). In some aspects, the CCL20 promoter of the present disclosure (i.e., a CCL20 promoter of SEQ ID NO:1-5 or a functional fragment or functional variant thereof) is stimulated with granulocyte-macrophage colony stimulating factor (GM-CSF). In some aspects, the CCL20 promoter of the present disclosure (i.e., a CCL20 promoter of any one of SEQ ID NOS:1-5 or a functional fragment or functional variant thereof) is stimulated with soluble interleukin 6 receptor (sIL-6R). In some aspects, the CCL20 promoter of the present disclosure (i.e., a CCL20 promoter of any one of SEQ ID NOS:1-5 or a functional fragment or functional variant thereof) is stimulated with interleukin 17 (IL-17). In some aspects, the CCL20 promoter of the present disclosure (i.e., a CCL20 promoter of any one of SEQ ID NOS:1-5 or a functional fragment or functional variant thereof) is stimulated with CD30.

In some aspects, the polynucleotide comprises a CCL20 promoter comprising, consisting, or consisting essentially of the sequence set forth in SEQ ID NO: 1. In some aspects, the polynucleotide comprises a CCL20 promoter comprising, consisting, or consisting essentially of the sequence set forth in SEQ ID NO: 2. In some aspects, the polynucleotide comprises a CCL20 promoter comprising, consisting, or consisting essentially of the sequence set forth in SEQ ID NO: 3. In some aspects, the polynucleotide comprises a CCL20 promoter comprising, consisting, or consisting essentially of the sequence set forth in SEQ ID NO: 4. In some aspects, the polynucleotide comprises a CCL20 promoter comprising, consisting, or consisting essentially of the sequence set forth in SEQ ID NO: 5.

(CLL20 promoter full sequence)
SEQ ID NO: 1
GAGAGTTCTTATACTGCCTTAGGTTAGGGATTCATATTACATTATCATCATTAGTACTGTTATTTGACATTTGCTGTG
CTGACTAGCTACTGCTGATAGGTTTTCTTTCCTCAACAATTCTGAGGCTCTATATTGAGTTATATTAGTACATCATCA
TGGAGAGTTAAAGGTAGGTAAGGATTATTTTCTGAACTGCAATATTGATTAAAGCCATGTGAATGTATAAGATTCTTA
GAAGAGTTGACATTAAATCAAGGTGAAGCTGAGGTTTGAGCCTTACTTAAAGGCTGATATTTTCCACTCTAACTGCGG
ACAGTACTGTAGCACTGTTATAGTACCTGCTCTGAATGTTAGTCTAGCAACTCAGGGTCTTCTTCATGACAGCTGAAC
CTCAACCATGTGATGGTAAATGTGTAGCAGAGTATGCCTGGCATCCCACCTGCTCCTCCTCCCCCTCCTCCTTGACTG
GTTCTGGAAAGCAAATAGGGTGTAACAATAGGAGTTCTGGAATGTTCCTGTGTGGGGCTGACCTTTGTATCGCTGTTA
ATCCTCTATTTTCAGACACAAAAATGATTAAGTTAAAACTGGATGAAAGTCTTTTCTGGGTCACAGGGCTGAGCTGCT
TTTGCTCTTTGCAAATACAAAGAATTTAACAGGATTCTCCCCTTCTCAACTTCCTGTCCCCCACCCTGACCTTCGCAC
CTTCCCAATATGAGGAAAAAGCAGGAAGTTTTCCTTGCGGGTTTTTTTTATGATGACATGATGGGGCCAGTTGATCAA
TGGGGAAAACCCCATGTGGCAACACGCCTTCTGTGTACATTCCCAATATTTGCTATAAATAGGGCCATCCCAGGCTGC
TGTCAGAATATAACAGCACTCCCAAAGAACTGGGTACTCAACACTGAGCAGATCTGTTCTTTGA
(CCL20 promoter without 5′ region)
SEQ ID NO: 2
GTTATTTGACATTTGCTGTGCTGACTAGCTACTGCTGATAGGTTTTCTTTCCTCAACAATTCTGAGGCTCTATATTGA
GTTATATTAGTACATCATCATGGAGAGTTAAAGGTAGGTAAGGATTATTTTCTGAACTGCAATATTGATTAAAGCCAT
GTGAATGTATAAGATTCTTAGAAGAGTTGACATTAAATCAAGGTGAAGCTGAGGTTTGAGCCTTACTTAAAGGCTGAT
ATTTTCCACTCTAACTGCGGACAGTACTGTAGCACTGTTATAGTACCTGCTCTGAATGTTAGTCTAGCAACTCAGGGT
CTTCTTCATGACAGCTGAACCTCAACCATGTGATGGTAAATGTGTAGCAGAGTATGCCTGGCATCCCACCTGCTCCTC
CTCCCCCTCCTCCTTGACTGGTTCTGGAAAGCAAATAGGGTGTAACAATAGGAGTTCTGGAATGTTCCTGTGTGGGGC
TGACCTTTGTATCGCTGTTAATCCTCTATTTTCAGACACAAAAATGATTAAGTTAAAACTGGATGAAAGTCTTTTCTG
GGTCACAGGGCTGAGCTGCTTTTGCTCTTTGCAAATACAAAGAATTTAACAGGATTCTCCCCTTCTCAACTTCCTGTC
CCCCACCCTGACCTTCGCACCTTCCCAATATGAGGAAAAAGCAGGAAGTTTTCCTTGCGGGTTTTTTTTATGATGACA
TGATGGGGCCAGTTGATCAATGGGGAAAACCCCATGTGGCAACACGCCTTCTGTGTACATTCCCAATATTTGCTATAA
ATAGGGCCATCCCAGGCTGCTGTCAGAATATAACAGCACTCCCAAAGAACTGGGTACTCAACACTGAGCAGATCTGTT
CTTTGA
(CCL20 promoter without 3′ region)
SEQ ID NO: 3
GAGAGTTCTTATACTGCCTTAGGTTAGGGATTCATATTACATTATCATCATTAGTACTGTTATTTGACATTTGCTGTG
CTGACTAGCTACTGCTGATAGGTTTTCTTTCCTCAACAATTCTGAGGCTCTATATTGAGTTATATTAGTACATCATCA
TGGAGAGTTAAAGGTAGGTAAGGATTATTTTCTGAACTGCAATATTGATTAAAGCCATGTGAATGTATAAGATTCTTA
GAAGAGTTGACATTAAATCAAGGTGAAGCTGAGGTTTGAGCCTTACTTAAAGGCTGATATTTTCCACTCTAACTGCGG
ACAGTACTGTAGCACTGTTATAGTACCTGCTCTGAATGTTAGTCTAGCAACTCAGGGTCTTCTTCATGACAGCTGAAC
CTCAACCATGTGATGGTAAATGTGTAGCAGAGTATGCCTGGCATCCCACCTGCTCCTCCTCCCCCTCCTCCTTGACTG
GTTCTGGAAAGCAAATAGGGTGTAACAATAGGAGTTCTGGAATGTTCCTGTGTGGGGCTGACCTTTGTATCGCTGTTA
ATCCTCTATTTTCAGACACAAAAATGATTAAGTTAAAACTGGATGAAAGTCTTTTCTGGGTCACAGGGCTGAGCTGCT
TTTGCTCTTTGCAAATACAAAGAATTTAACAGGATTCTCCCCTTCTCAACTTCCTGTCCCCCACCCTGACCTTCGCAC
CTTCCCAATATGAGGAAAAAGCAGGAAGTTTTCCTTGCGGGTTTTTTTTATGATGACATGATGGGGCCAGTTGATCAA
TGGGGAAAACCCCATGTGGCAACACGCCTTCTGTGTACATTCCCAATATTTGCTATAAATAGGGCCATCCCAGGCTGC
TGTCAGAATATAACAG
(CCL20 promoter, core region)
SEQ ID NO: 4
GTTATTTGACATTTGCTGTGCTGACTAGCTACTGCTGATAGGTTTTCTTTCCTCAACAATTCTGAGGCTCTATATTGA
GTTATATTAGTACATCATCATGGAGAGTTAAAGGTAGGTAAGGATTATTTTCTGAACTGCAATATTGATTAAAGCCAT
GTGAATGTATAAGATTCTTAGAAGAGTTGACATTAAATCAAGGTGAAGCTGAGGTTTGAGCCTTACTTAAAGGCTGAT
ATTTTCCACTCTAACTGCGGACAGTACTGTAGCACTGTTATAGTACCTGCTCTGAATGTTAGTCTAGCAACTCAGGGT
CTTCTTCATGACAGCTGAACCTCAACCATGTGATGGTAAATGTGTAGCAGAGTATGCCTGGCATCCCACCTGCTCCTC
CTCCCCCTCCTCCTTGACTGGTTCTGGAAAGCAAATAGGGTGTAACAATAGGAGTTCTGGAATGTTCCTGTGTGGGGC
TGACCTTTGTATCGCTGTTAATCCTCTATTTTCAGACACAAAAATGATTAAGTTAAAACTGGATGAAAGTCTTTTCTG
GGTCACAGGGCTGAGCTGCTTTTGCTCTTTGCAAATACAAAGAATTTAACAGGATTCTCCCCTTCTCAACTTCCTGTC
CCCCACCCTGACCTTCGCACCTTCCCAATATGAGGAAAAAGCAGGAAGTTTTCCTTGCGGGTTTTTTTTATGATGACA
TGATGGGGCCAGTTGATCAATGGGGAAAACCCCATGTGGCAACACGCCTTCTGTGTACATTCCCAATATTTGCTATAA
ATAGGGCCATCCCAGGCTGCTGTCAGAATATAACAG
(CCL promoter, small)
SEQ ID NO: 5
CCCACCTGCTCCTCCTCCCCCTCCTCCTTGACTGGTTCTGGAAAGCAAATAGGGTGTAACAATAGGAGTTCTGGAATG
TTCCTGTGTGGGGCTGACCTTTGTATCGCTGTTAATCCTCTATTTTCAGACACAAAAATGATTAAGTTAAAACTGGAT
GAAAGTCTTTTCTGGGTCACAGGGCTGAGCTGCTTT

In some aspects, the CCL20 promoter of the present disclosure is a functional variant of a CCL20 promoter having the sequence set forth in any one of SEQ ID NOS: 1, 2, 3, 4 or 5. In some aspects, the CCL20 promoter of the present disclosure is a functional fragment of a CCL20 promoter having the sequence set forth in any one of SEQ ID NOS: 1, 2, 3, 4 or 5. As defined above, a functional fragment can be a truncated from of a CCL20 promoter of any one of SEQ ID NOS: 1, 2, 3, 4 or 5, wherein the CCL20 functional fragment promoter is still capable of inducing the expression of a heterologous protein encoded by a heterologous gene operatively linked to the CCL20 promoter in response to stimulation by a cytokine, e.g., IL-1α. Similarly, a functional variant can be a mutant of a CCL20 promoter of any one of SEQ ID NOS: 1, 2, 3, 4 or 5, wherein the CCL20 functional variant promoter is still capable of inducing the expression of a heterologous protein encoded by a heterologous gene operatively linked to the CCL20 promoter in response to stimulation by a cytokine, e.g., IL-1α. In some aspects, the promoter activity of a functional fragment or functional variant is determined using a heterologous gene that is a reporter gene, e.g., luciferase.

In some aspects, the CCL20 functional fragment or functional variant promoter has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the activity of a CCL20 promoter of any one of SEQ ID NOS: 1, 2, 3, 4 or 5 under the same experimental conditions.

In some aspects, the CCL20 functional fragment or functional variant promoter has about 20%, about 25%, about 30%, about 35%, about 40%, about 45%, about 50%, about 55%, about 60%, about 65%, about 70%, about 75%, about 80%, about 85%, about 90%, about 95%, or about 100% of the activity of a CCL20 promoter of any one of SEQ ID NOS: 1, 2, 3, 4 or 5 under the same experimental conditions.

In some aspects, the CCL20 functional fragment or functional variant promoter has about 20% to about 30%, about 25% to about 35%, about 30% to 40%, about 35% to about 45%, about 40% to about 50%, about 45% to about 55%, about 50% to about 60%, about 55% to about 65%, about 60% to about 70%, about 65% to about 75%, about 70% to about 80%, about 75% to about 85%, about 80% to about 90%, about 85% to about 95%, about or 90% to about 100% of the activity of a CCL20 promoter of any one of SEQ ID NOS: 1, 2, 3, 4 or 5 under the same experimental conditions.

In some aspects, the CCL20 functional fragment or functional variant promoter is a gain-of-function promoter, i.e., the activity of the CCL20 functional fragment or functional variant promoter is higher than that of a CCL20 promoter of any one of SEQ ID NOS: 1, 2, 3, 4 or 5 under the same experimental conditions.

In some aspects, the gain-of-function CCL20 promoter has an activity that is at least about 110%, at least about 120%, at least about 130%, at least about 140%, at least about 150%, at least about 160%, at least about 170%, at least about 180%, at least about 190%, or at least about 200% of the activity of a CCL20 promoter of any one of SEQ ID NOS: 1, 2, 3, 4 or 5 under the same experimental conditions.

In some aspects, the gain-of-function CCL20 promoter has an activity that is at least about 2-fold, at least about 3-fold, at least about 4-fold, at least about 5-fold, at least about 6-fold, at least about 7-fold, at least about 8-fold, at least about 9-fold, or at least about 10-fold the activity of a CCL20 promoter of any one of SEQ ID NOS: 1, 2, 3, 4 or 5 under the same experimental conditions.

In some aspects, the gain-of-function CCL20 promoter has an activity that is about 2-fold, about 3-fold, about 4-fold, about 5-fold, about 6-fold, about 7-fold, about 8-fold, about 9-fold, or about 10-fold the activity of a CCL20 promoter of any one of SEQ ID NOS: 1, 2, 3, 4 or 5 under the same experimental conditions.

In some aspects, the gain-of-function CCL20 promoter has an activity that is about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, or about 9-fold to about 10-fold the activity of a CCL20 promoter of any one of SEQ ID NOS: 1, 2, 3, 4 or 5 under the same experimental conditions.

In some aspects, the CCL20 promoter of the present disclosure has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to a CCL20 promoter sequence set forth in any one of SEQ ID NOS: 1, 2, 3, 4 or 5, wherein the CCL20 promoter is capable of inducing the expression of a heterologous protein in response to stimulation by a cytokine, e.g., IL-1α.

In some aspects, the CCL20 promoter of the present disclosure has about 60%, about 65%, about 70%, about 75%, about 80%, about 85%, about 90%, about 95%, about 96%, about 97%, about 98%, or about 99% sequence identity to a CCL20 promoter sequence set forth in any one of SEQ ID NOS: 1, 2, 3, 4 or 5, wherein the CCL20 promoter is capable of inducing the expression of a heterologous protein in response to stimulation by a cytokine, e.g., IL-1α.

In some aspects, the CCL20 promoter of the present disclosure has between about 60% and about 65%, between about 65% and about 70%, between about 70% and about 75%, between about 75% and about 80%, between about 80% and about 85%, between about 85% and about 90%, between about 90% and about 95%, between about 95% and about 100% sequence identity to a CCL20 promoter sequence set forth in any one of SEQ ID NOS: 1, 2, 3, 4 or 5, wherein the CCL20 promoter is capable of inducing the expression of a heterologous protein in response to stimulation by a cytokine, e.g., IL-1α.

In some aspects, the CCL20 promoter is a functional fragment of a CCL20 promoter having a sequence set forth in any one of SEQ ID NOS: 1, 2, 3, 4 or 5, wherein the CCL20 functional fragment promoter is capable of inducing the expression of a heterologous protein in response to stimulation by a cytokine, e.g., IL-1α.

In some aspects, the CCL20 promoter is a functional fragment of a CCL20 promoter having a sequence set forth in any one of SEQ ID NOS: 1, 2, 3, 4 or 5, wherein the CCL20 functional fragment promoter comprises a truncation of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, or at least about 600 nucleotides from the 5′ of a CCL20 promoter sequence set forth in any one of SEQ ID NOS: 1, 2, 3, 4 or 5, wherein the CCL20 functional fragment promoter is capable of inducing the expression of a heterologous protein in response to stimulation by a cytokine, e.g., IL-1α.

In some aspects, the CCL20 promoter is a functional fragment of a CCL20 promoter having a sequence set forth in any one of SEQ ID NOS: 1, 2, 3, 4 or 5, wherein the CCL20 functional fragment promoter comprises a truncation of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, or about 600 nucleotides from the 5′ of a CCL20 promoter sequence set forth in any one of SEQ ID NOS: 1, 2, 3, 4 or 5, wherein the CCL20 functional fragment promoter is capable of inducing the expression of a heterologous protein in response to stimulation by a cytokine, e.g., IL-1α.

In some aspects, the CCL20 promoter is a functional fragment of a CCL20 promoter having a sequence set forth in any one of SEQ ID NOS: 1, 2, 3, 4 or 5, wherein the CCL20 functional fragment promoter comprises a truncation of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, or at least about 600 nucleotides from the 3′ of a CCL20 promoter sequence set forth in any one of SEQ ID NOS: 1, 2, 3, 4 or 5, wherein the CCL20 functional fragment promoter is capable of inducing the expression of a heterologous protein in response to stimulation by a cytokine, e.g., IL-1α.

In some aspects, the CCL20 promoter is a functional fragment of a CCL20 promoter having a sequence set forth in any one of SEQ ID NOS: 1, 2, 3, 4 or 5, wherein the CCL20 functional fragment promoter comprises a truncation of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, or about 600 nucleotides from the 3′ of a CCL20 promoter sequence set forth in any one of SEQ ID NOS: 1, 2, 3, 4 or 5, wherein the CCL20 functional fragment promoter is capable of inducing the expression of a heterologous protein in response to stimulation by a cytokine, e.g., IL-1α.

In some aspects, the CCL20 promoter is a functional fragment of a CCL20 promoter having a sequence set forth in any one of SEQ ID NOS: 1, 2, 3, 4 or 5, wherein the CCL20 functional fragment promoter comprises an internal truncation (i.e., a truncation that does not comprise the 5′ or the 3′ end of the sequence) of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, or at least about 600 nucleotides from a CCL20 promoter sequence set forth in any one of SEQ ID NOS: 1, 2, 3, 4 or 5, wherein the CCL20 functional fragment promoter is capable of inducing the expression of a heterologous protein in response to stimulation by a cytokine, e.g., IL-1α.

In some aspects, the CCL20 promoter is a functional fragment of a CCL20 promoter having a sequence set forth in any one of SEQ ID NOS: 1, 2, 3, 4 or 5, wherein the CCL20 functional fragment promoter comprises an internal truncation (i.e., a truncation that does not comprise the 5′ or the 3′ end of the sequence) of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, or about 600 nucleotides from a CCL20 promoter sequence set forth in any one of SEQ ID NOS: 1, 2, 3, 4 or 5, wherein the CCL20 functional fragment promoter is capable of inducing the expression of a heterologous protein in response to stimulation by a cytokine, e.g., IL-1α.

In some aspects, the CCL20 promoter is a functional variant of a CCL20 promoter having a sequence set forth in any one of SEQ ID NOS: 1, 2, 3, 4 or 5, wherein the CCL20 functional variant promoter is a mutant capable of inducing the expression of a heterologous protein in response to stimulation by a cytokine, e.g., IL-1α. As used herein the term mutant refers to a CCL20 functional variant promoter comprising one or more nucleotide substitutions, insertions, or deletions with respect to a CCL20 promoter sequence set forth in any one of SEQ ID NOS: 1, 2, 3, 4 or 5, wherein the CCL20 functional variant promoter is capable of inducing the expression of a heterologous protein in response to stimulation by a cytokine, e.g., IL-1α.

In some aspects, the CCL20 functional variant promoter comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 substitutions. In some aspects, the CCL20 functional variant promoter comprises at least about 20, at least about 25, at least about 30, at least about 35, at least about 40, at least about 45 or at least about 50 substitutions. In some aspects, the CCL20 functional variant promoter comprises 1, 2, 3, 4, 5, 6, 7, 8, 9, 10, 11, 12, 13, 14, 15, 16, 17, 18, 19, or 20 substitutions. In some aspects, the CCL20 functional variant promoter comprises about 20, about 25, about 30, about 35, about 40, about 45 or about 50 substitutions.

In some aspects, the CCL20 functional variant promoter comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more deletions. In some aspects, the CCL20 functional variant promoter comprises at least about 20, at least about 25, at least about 30, at least about 35, at least about 40, at least about 45 or at least about 50 deletions. In some aspects, the CCL20 functional variant promoter comprises 1, 2, 3, 4, 5, 6, 7, 8, 9, 10, 11, 12, 13, 14, 15, 16, 17, 18, 19, or 20 deletions. In some aspects, the CCL20 functional variant promoter comprises about 20, about 25, about 30, about 35, about 40, about 45 or about 50 deletions.

In some aspects, the CCL20 functional variant promoter comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more insertions. In some aspects, the CCL20 functional variant promoter comprises at least about 20, at least about 25, at least about 30, at least about 35, at least about 40, at least about 45 or at least about 50 insertions. In some aspects, the CCL20 functional variant promoter comprises 1, 2, 3, 4, 5, 6, 7, 8, 9, 10, 11, 12, 13, 14, 15, 16, 17, 18, 19, or 20 insertions. In some aspects, the CCL20 functional variant promoter comprises about 20, about 25, about 30, about 35, about 40, about 45 or about 50 insertions.

In some aspects, a mutation can comprise multiple nucleotides in a CCL20 promoter sequence set forth in any one of SEQ ID NOS: 1, 2, 3, 4 or 5, e.g., a subsequence comprising more than one nucleotides can be inserted, deleted, or substituted. In some aspects, a mutation comprise a single nucleotide change in a CCL20 promoter sequence set forth in any one of SEQ ID NOS: 1, 2, 3, 4, or 5, i.e., the mutation is a point mutation. In some aspects, the sequence of the CCL20 promoter of the present disclosure has 1, 2, 3, 4, 5, 6, 7, 8, 9, 10, 11, 12, 13, 14, 15, 16, 17, 18, 19, 20, or more than 20 point mutations with respect to a CCL20 promoter sequence set forth in any one of SEQ ID NOS: 1, 2, 3, 4 or 5. In some aspects, the sequence of the CCL20 promoter of the present disclosure has at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 point mutations with respect to a CCL20 promoter sequence set forth in any one of SEQ ID NOS: 1, 2, 3, 4 or 5. In some aspects, the point mutations are conservative point mutations. In some aspects, the point mutations are non-conservative point mutations. In some aspects, the point mutations comprise both conservative and non-conservative point mutations.

In some aspects, a CCL20 promoter of the present disclosure comprises at least about 50 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 550 nucleotides, at least about 600 nucleotides, at least about 650 nucleotides, at least about 700 nucleotides, at least about 750 nucleotides, at least about 800 nucleotides, at least about 850 nucleotides, or at least about 900 nucleotides from the sequence set forth in SEQ ID NO: 1, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises at least about 50 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 550 nucleotides, at least about 600 nucleotides, at least about 650 nucleotides, at least about 700 nucleotides, at least about 750 nucleotides, at least about 800 nucleotides, at least about 850 nucleotides, or at least about 900 nucleotides from the sequence set forth in SEQ ID NO: 2, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises at least about 50 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 550 nucleotides, at least about 600 nucleotides, at least about 650 nucleotides, at least about 700 nucleotides, at least about 750 nucleotides, at least about 800 nucleotides, at least about 850 nucleotides, or at least about 900 nucleotides from the sequence set forth in SEQ ID NO: 3, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises at least about 50 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 550 nucleotides, at least about 600 nucleotides, at least about 650 nucleotides, at least about 700 nucleotides, at least about 750 nucleotides, at least about 800 nucleotides, at least about 850 nucleotides, or at least about 900 nucleotides from the sequence set forth in SEQ ID NO: 4, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises at least about 50 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 550 nucleotides, at least about 600 nucleotides, at least about 650 nucleotides, at least about 700 nucleotides, at least about 750 nucleotides, at least about 800 nucleotides, at least about 850 nucleotides, or at least about 900 nucleotides from the sequence set forth in SEQ ID NO: 5, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises about 50 nucleotides, about 100 nucleotides, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, about 600 nucleotides, about 650 nucleotides, about 700 nucleotides, about 750 nucleotides, about 800 nucleotides, about 850 nucleotides, or about 900 nucleotides from the sequence set forth in SEQ ID NO: 1, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises about 50 nucleotides, about 100 nucleotides, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, about 600 nucleotides, about 650 nucleotides, about 700 nucleotides, about 750 nucleotides, about 800 nucleotides, about 850 nucleotides, or about 900 nucleotides from the sequence set forth in SEQ ID NO: 2, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises about 50 nucleotides, about 100 nucleotides, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, about 600 nucleotides, about 650 nucleotides, about 700 nucleotides, about 750 nucleotides, about 800 nucleotides, about 850 nucleotides, or about 900 nucleotides from the sequence set forth in SEQ ID NO: 3, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises about 50 nucleotides, about 100 nucleotides, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, about 600 nucleotides, about 650 nucleotides, about 700 nucleotides, about 750 nucleotides, about 800 nucleotides, about 850 nucleotides, or about 900 nucleotides from the sequence set forth in SEQ ID NO: 4, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises about 50 nucleotides, about 100 nucleotides, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, about 600 nucleotides, about 650 nucleotides, about 700 nucleotides, about 750 nucleotides, about 800 nucleotides, about 850 nucleotides, or about 900 nucleotides from the sequence set forth in SEQ ID NO: 5, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure comprises between about 50 nucleotides and about 100 nucleotides, between about 100 nucleotides and about 150 nucleotides, between about 150 nucleotides and about 200 nucleotides, between about 200 nucleotides and about 250 nucleotides, between about 250 nucleotides and about 300 nucleotides, between about 300 nucleotides and about 350 nucleotides, between about 350 nucleotides and about 400 nucleotides, between about 400 nucleotides and about 450 nucleotides, between about 450 nucleotides and about 500 nucleotides, between about 500 nucleotides and about 550 nucleotides, between about 550 nucleotides and about 600 nucleotides, between about 600 nucleotides and about 650 nucleotides, between about 650 nucleotides and about 700 nucleotides, between about 700 nucleotides and about 750 nucleotides, between about 750 nucleotides and about 800 nucleotides, between about 800 nucleotides and about 850 nucleotides, between about 850 nucleotides and about 900 nucleotides, between about 100 nucleotides and about 200 nucleotides, between about 200 nucleotides and about 300 nucleotides, between about 300 nucleotides and about 400 nucleotides, between about 400 nucleotides and about 500 nucleotides, between about 500 nucleotides and about 600 nucleotides, between about 600 nucleotides and about 700 nucleotides, between about 700 nucleotides and about 800 nucleotides, between about 800 nucleotides and about 900 nucleotides, between about 100 nucleotides and about 300 nucleotides, between about 200 nucleotides and about 400 nucleotides, between about 300 nucleotides and about 500 nucleotides, between about 400 nucleotides and about 600 nucleotides, between about 500 nucleotides and about 700 nucleotides, between about 600 nucleotides and about 800 nucleotides, between about 700 nucleotides and about 900 nucleotides, between about 100 nucleotides and about 400 nucleotides, between about 200 nucleotides and about 500 nucleotides, between about 300 nucleotides and about 600 nucleotides, between about 400 nucleotides and about 700 nucleotides, between about 500 nucleotides and about 800, or between about 600 nucleotides and about 900 nucleotides from a sequence set forth in any one of SEQ ID NOS: 1, 2, 3, 4 or 5, wherein the CCL20 promoter can induce the expression of a protein or nucleic acid encoded by a heterologous gene operatively linked to the promoter in response to stimulation with a cytokine, e.g., IL-1α.

In some aspects, a CCL20 promoter of the present disclosure is about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, or about 900 nucleotides in length. In some aspects, a CCL20 promoter of the present disclosure is at least about 100, at least about 125, at least about 150, at least about 175, at least about 200, at least about 225, at least about 250, at least about 275, at least about 300, at least about 325, at least about 350, at least about 375, at least about 400, at least about 425, at least about 450, at least about 475, at least about 500, at least about 525, at least about 550, at least about 575, at least about 600, at least about 625, at least about 650, at least about 675, at least about 700, at least about 725, at least about 750, at least about 775, at least about 800, at least about 825, at least about 850, at least about 875, or at least about 900 nucleotides in length.

In some aspects, the heterologous gene encodes a nucleic acid. In some aspects, the nucleic acid is an endogenous nucleic acid, e.g., a miRNA. In some aspects, the nucleic acid is an exogenous nucleic acid, e.g., an RNA interference agent such as an antisense oligonucleotide. In some aspects, the heterologous gene encodes a protein, e.g., is a therapeutic protein. In some aspects, the therapeutic protein is, e.g., a chimeric antigenic receptor (CAR), an antibody, a tumor suppressor, an inducer of apoptosis, an enzyme, a hormone, or any combination thereof.

In some aspects, the therapeutic protein comprises an anti-inflammatory antibody or an antigen-binding portion thereof. In some aspects, the anti-inflammatory antibody or antigen-binding portion thereof inhibits TNF-α. In some aspects, the anti-inflammatory antibody or antigen-binding portion thereof inhibits IL-6. In some aspects, the anti-inflammatory antibody or antigen-binding portion thereof inhibits IL-12. In some aspects, the anti-inflammatory antibody or antigen-binding portion thereof inhibits IL-23. In some aspects, the anti-inflammatory antibody or antigen-binding portion thereof inhibits IL-12 and IL-23. In some aspects, the anti-inflammatory antibody or antigen-binding portion thereof inhibits IL-2. In some aspects, the anti-inflammatory antibody or antigen-binding portion thereof inhibits immunoglobulin E (IgE). In some aspects, the anti-inflammatory antibody or antigen-binding portion thereof inhibits interleukin 6 receptor (IL-6R). In some aspects, the anti-inflammatory antibody or antigen-binding portion thereof inhibits IL-17A. In some aspects, the anti-inflammatory antibody or antigen-binding portion thereof inhibits IL-5. In some aspects, the anti-inflammatory antibody or antigen-binding portion thereof inhibits IL-4RA. In some aspects, the anti-inflammatory antibody or antigen-binding portion thereof inhibits IL-1B. In some aspects, the anti-inflammatory antibody or antigen-binding portion thereof inhibits IL-17RA.

In some aspects, the anti-inflammatory antibody comprises infliximab or an antigen-binding portion thereof. In some aspects, the anti-inflammatory antibody comprises adalimumab or an antigen-binding portion thereof. In some aspects, the anti-inflammatory antibody comprises ustekinumab or an antigen-binding portion thereof. In some aspects, the anti-inflammatory antibody comprises basiliximab or an antigen-binding portion thereof. In some aspects, the anti-inflammatory antibody comprises daclizumab or an antigen-binding portion thereof. In some aspects, the anti-inflammatory antibody comprises omalizumab or an antigen-binding portion thereof. In some aspects, the anti-inflammatory antibody comprises tildrakizumab or an antigen-binding portion thereof. In some aspects, the anti-inflammatory antibody comprises guselkumab or an antigen-binding portion thereof. In some aspects, the anti-inflammatory antibody comprises sarilumab or an antigen-binding portion thereof. In some aspects, the anti-inflammatory antibody comprises tocilizumab or an antigen-binding portion thereof. In some aspects, the anti-inflammatory antibody comprises siltuximab or an antigen-binding portion thereof. In some aspects, the anti-inflammatory antibody comprises secukinumab or an antigen-binding portion thereof. In some aspects, the anti-inflammatory antibody comprises reslizumab or an antigen-binding portion thereof. In some aspects, the anti-inflammatory antibody comprises mepolizumab or an antigen-binding portion thereof. In some aspects, the anti-inflammatory antibody comprises dupilumab or an antigen-binding portion thereof. In some aspects, the anti-inflammatory antibody comprises certilizumab or an antigen-binding portion thereof. In some aspects, the anti-inflammatory antibody comprises canakinumab or an antigen-binding portion thereof. In some aspects, the anti-inflammatory antibody comprises brodalumab or an antigen-binding portion thereof.

In some aspects, the heterologous gene encodes a pro-inflammatory cytokine antagonist. In some aspects, the heterologous gene encodes an interleukin. In some aspects, the heterologous gene encodes an anti-inflammatory interleukin. In some aspects, the heterologous gene encodes an inflammatory interleukin antagonist. In some aspects, the heterologous gene encodes IL-1 receptor antagonist (IL-1RA). In some aspects, the heterologous gene encodes interleukin 36 receptor antagonist (IL-36RA). In some aspects, the heterologous gene encodes interleukin 37 (IL-37). In some aspects, the heterologous gene encodes IL-4. In some aspects, the heterologous gene encodes IL-6. In some aspects, the heterologous gene encodes IL-10. In some aspects, the heterologous gene encodes IL-11. In some aspects, the heterologous gene encodes IL-13. In some aspects, the heterologous gene encodes a specific cytokine receptor for IL-1. In some aspects, the heterologous gene encodes a specific receptor for TNF-α. In some aspects, the heterologous gene encodes a specific receptor for IL-18. In some aspects, the heterologous gene encodes an anti-inflammatory protein.

In some aspects, the heterologous gene encodes a protein or nucleic acid that can block the expression or inhibit the activity of a pro-inflammatory gene (i.e., a gene which, when expressed, promotes inflammation) or its protein product, for example, IL-1, IL-4, IL-5, IL-6, IL-8, IL-12, IL-13, IL-17, IL-21, IL-22, IL-23, IL-27, IFN, CCL2, CCL3, CCL5, CCL20, CXCL5, CXCL10, CXCL12, CXCL13, or TNF-α.

In some aspects, the heterologous gene encodes a protein or nucleic acid having a regulatory role in an inflammation pathway. In some aspects, the heterologous gene encodes a regulatory protein in an inflammation pathway. In some aspects, the heterologous gene encodes a regulatory protein or polypeptide that prevents the activation of a pro-inflammatory pathway. In some aspects the heterologous gene encodes, an inhibitor of an inflammatory mediator. Thus, in some aspects, the heterologous gene can encode a modulator or inhibitor of a protein known to be key mediator (targets) in inflammatory response pathways. Key mediator proteins that can be modulated directed or indirectly via a CCL20 promoter of the present disclosure (i.e., a CCL20 promoter of any one of SEQ ID NOS: 1-5 or a functional variant thereof) include, e.g., aggrecan, IFN-γ, LFA-1, Th1, angiopoietin-2, Mac-1, Th2, BDNF, MCP-1/CCL2, Th9, CD23, MCP-2/CCL8, Th17, CD30, MCP-3/CCL7, Th22, CD40L, MIG/CXCL9, TLR 1, C-Reactive Protein (CRP), IKK, MIP-1α/CCL3, TLR 2, CTL, IL-1, MIP-1β/CCL4, TLR 3, E-cadherin, IL-2, MIP-2, TLR 4, EGF, IL-3, NF-κB, TLR 5, ELAM-1, IL-4, PAI-1, TLR 6, Eotaxin/CCL11, IL-5, PDGF-BB, TLR 7, FGF basic, IL-6, PECAM-lg, TLR 8, G-CSF, IL-8, p-Selectin, TLR 9, GDNF, IL-10, RANTES/CCL5, TLR 10, GM-CSF, IL-12, SAA, TNF-α, GRO-α, IL-13, STAT1, TNF-β, HGF, IL-15, STAT2, TGF-β1, HIF-1α, IL-16, STAT3, TNF-RI, ICAM-1, IL-17, STAT4, TNF-RII, IFN-α, IL-18, STAT5, VCAM-1, IFN-β, IL-23, STAT6, or VEGF.

In some aspects, the heterologous gene encodes a chimeric antigen receptor (CAR). In some aspects, the heterologous gene encodes an enzyme, e.g., an enzyme that is desired to express at levels higher than endogenous normal levels as part of an enzyme replacement therapy. In some aspects, the heterologous gene encodes a hormone. In some aspects, the heterologous gene encodes a protein or nucleic acid that upon expression results in the decrease of systemic effects associated with inflammation such as fever, leukocytosis, leukopenia, vasodilation, septic shock, swelling, pain, loss of function, rubor (redness), necrosis, or any combination thereof.

In general, the CCL20 promoters disclosed herein (i.e., a CCL20 promoter of any one of SEQ ID NOS:1-5 or a functional fragment or functional variant thereof) can be used to elicit the expression of any therapeutic protein or therapeutic nucleic acid in a subject following administration of a vector comprising a CCL20 promoter disclosed herein operably linked to a gene encoding such protein or nucleic acid or a cell, e.g., a cell transiently or stably transfected with a vector comprising a CCL20 promoter disclosed herein operably linked to a gene encoding such protein or nucleic a gene, or a viral particle comprising a CCL20 promoter disclosed herein operably linked to a gene encoding such protein or nucleic. In some aspects, the therapeutic protein may be an antibody targeting cancer cells, or a coagulation factor. In some aspects, the heterologous gene encodes a reporter protein, e.g., luciferase, green fluorescent protein, red fluorescent protein, beta-galactosidase.

In some aspects, the present disclosure provides a vector comprising a promoter region comprising a CCL20 promoter comprising a sequence set forth in any one of SEQ ID NOS: 1-5 or a functional fragment or functional variant thereof. In some aspects, the vector comprises a heterologous gene operably linked to the CCL20 promoter. In some aspects, the vector does not comprise a heterologous gene operably linked to the CCL20 promoter. In some aspects, the CCL20 promoter is a functional fragment of a sequence set forth in any one of SEQ ID NOS: 1-5. In some aspects, the functional fragment has a 5′ truncation, a 3′ truncation, or both relative to a CCL20 promoter sequence set forth in any one of SEQ ID NOS: 1-5. In some aspects, the CCL20 promoter is a functional variant having a sequence with at least about 70%, about 75%, about 80%, about 85%, about 90%, about 95%, about 96%, about 97%, about 98%, or about 99% sequence identity to the sequence set forth in any one of SEQ ID NOS:1-5. In some aspects, the sequence of the CCL20 promoter functional variant has 1, 2, 3, 4, 5, 6, 7, 8, 9, 10, 11, 12, 13, 14, 15, 16, 17, 18, 19, or 20 point mutations with respect to a sequence set forth in any one of SEQ ID NOS: 1-5. In some aspects, the point mutations are conservative point mutations.

The present disclosure also provides a composition comprising:
(a) polynucleotide comprising a promoter region comprising a CCL20 promoter of the present disclosure, i.e., a promoter of any one of SEQ ID NOS:1-5 or a functional fragment or functional variant thereof, operatively linked to a heterologous gene (encoding, e.g., a therapeutic protein or therapeutic nucleic acid), wherein the CCL20 promoter induces expression of the heterologous gene in response to stimulation with a cytokine, e.g., IL-α.
(b) a delivery vehicle for delivery of the polynucleotide into a cell in a subject.

In some aspects the delivery vehicle comprises a nucleic acid, a plasmid, a vector (e.g., a viral vector), a prokaryotic cell, a eukaryotic cell, a lipid, or any combination thereof. In some aspects, the delivery vehicle comprises a lipid in a lipid vesicle, for example, a micelle, a liposome (e.g., a small unilamellar vesicle), a lipid particle (e.g., a lipid nanoparticle), or an extracellular vesicle (e.g., an exosome). In some aspects, the delivery vehicle comprises a viral particle. In some aspects, the delivery vehicle comprises a viral vector selected from the group consisting of adeno-associated virus, adenovirus, retrovirus, orthomyxovirus, paramyxovirus, papovavirus, picornavirus, lentivirus, herpes simplex virus, vaccinia virus, poxvirus, and alphavirus. In some aspects, the delivery vehicle comprises a prokaryotic cell. In some aspects, the delivery vehicle comprises a eukaryotic cell.

The present disclosure also provides a method of producing a cell population that expresses at least one heterologous gene under the control of a CCL20 promoter of the present disclosure (i.e., a CCL20 promoter of any one of SEQ ID NOS:1-5 or a functional fragment or functional variant thereof) comprising transfecting a cell with a composition comprising a polynucleotide comprising a promoter region comprising a CCL20 promoter of the present disclosure (i.e., a CCL20 promoter of any one of SEQ ID NOS:1-5 or a functional fragment or functional variant thereof) operatively linked to the heterologous gene, wherein the CCL20 promoter induces expression of the heterologous gene in response to stimulation with a cytokine, e.g., IL-α.

In some aspect, the cell population is prepared for administration to a subject in need thereof. In other aspects, the cell population is prepared for recombinant protein production in cell culture. In some aspects, the cell is a human cell. In some aspects, the cell is a human or non-human cell line suitable for protein production in cell cultures, e.g., CHO cells.

The present disclosure also provides a cell genetically modified to express a heterologous gene, wherein the cells comprise a polynucleotide disclosed herein, e.g., a polynucleotide comprising a promoter region comprising a CCL20 promoter of the present disclosure (i.e., a CCL20 promoter of any one of SEQ ID NOS:1-5 or a functional fragment or functional variant thereof) operatively linked to the heterologous gene, wherein the CCL20 promoter induces expression of the heterologous gene in response to stimulation with a cytokine, e.g., IL-α.

In some aspects, the cell can be inserted in a device, e.g., an implantable device. In some aspects, the cells are microencapsulated. In some aspects, the cell as microencapsulated in alginate microcapsules. An advantage of this approach with respect to traditional gene therapy is that the cells act as prosthetic devices, which do not require any host genome modification.

In other aspects, the cells are implanted using alternative cell implantation technologies such as biocompatible hollow fibers, which may enable an easy removal of the implanted device.

The present disclosure also provides a pharmaceutical composition comprising a polynucleotide, a composition, or a cell disclosed herein and a pharmaceutically acceptable carrier or excipient. Pharmaceutically acceptable excipients or carriers are determined in part by the particular composition being administered, as well as by the particular method used to administer the composition.

The present disclosure provides also a method of inducing, regulating, or enhancing the expression of a heterologous gene in a subject comprising (a) administering a polynucleotide, a composition, a cell, or a pharmaceutical composition disclosed herein comprising the heterologous gene under the control of a CCL20 promoter of the present disclosure (i.e., a CCL20 promoter of any one of SEQ ID NOS:1-5 or a functional fragment or functional variant thereof) to the subject; and (b) stimulating the CCL20 promoter with a cytokine, e.g., IL-α.

Also provided is a method of dose-dependent gene expression of a heterologous gene in a cell comprising (a) contacting the cell with a polynucleotide, a composition, a cell, or a pharmaceutical composition disclosed herein comprising a CCL20 promoter of the present disclosure (i.e., a CCL20 promoter of any one of SEQ ID NOS:1-5 or a functional fragment or functional variant thereof) operatively linked to the heterologous gene; and (b) contacting the cell with an effective amount of a cytokine, e.g., IL-α, capable of stimulating the CCL20 promoter to induce the expression of the heterologous gene in a dose-dependent manner.

The present disclosure also provides a method of controlling or inducing gene expression of a heterologous gene comprising operatively coupling a CCL20 promoter of the present disclosure (i.e., a CCL20 promoter of any one of SEQ ID NOS:1-5 or a functional fragment or functional variant thereof) to the heterologous gene, wherein the stimulation of the CCL20 promoter with a cytokine, e.g., IL-α, controls or induces the expression of the heterologous gene in response to the stimulation. Also provided a method of treating a disease in a subject, comprising administering an effective amount of a polynucleotide, composition, a cell, or a pharmaceutical composition disclosed herein to the subject. In some aspects, the administration of a construct comprising a CCL20 promoter of the present disclosure (i.e., a CCL20 promoter of any one of SEQ ID NOS:1-5 or a functional fragment or functional variant thereof) operably linked to a heterologous gene to a subject comprises transfecting cells from the subject, or cells from a donor with the construct. Transfection of the cells with the construct can be performed using any molecular biology methods known in the art. In some aspects, the construct is inserted in the genome of the cell using a nuclease system, e.g., CRISPR/Cas9.

The present disclosure also provides methods to control inflammation in a subject in need thereof comprising operatively coupling a CCL20 promoter of the present disclosure (i.e., a CCL20 promoter of any one of SEQ ID NOS:1-5 or a functional fragment or functional variant thereof) to a heterologous gene, wherein the stimulation of the CCL20 promoter with a cytokine, e.g., IL-1α, IL-1β, TNF (TNF-β or TNF-β), IL-6, sIL-6R, IL-17, or CD30, induces the expression of the heterologous gene in response to the stimulation, wherein the heterologous gene decreases immune activation directly, or indirectly via increase or decrease in the expression of another gene (e.g., a modulator gene in an inflammation pathway). In some aspects, the expression of the heterologous gene induced by the CCL20 promoter functionally results in the decrease of systemic effects associated with inflammation. For example, it can reduce fever, leukocytosis, leukopenia, vasodilation, septic shock, swelling, pain, loss of function, rubor (redness), necrosis, or any combination thereof.

As discussed above, the cytokine used to stimulate a CCL20 promoter of the present disclosure (i.e., a CCL20 promoter of any one of SEQ ID NOS:1-5 or a functional fragment or functional variant thereof) can be exogenous (i.e., the source of the cytokine is an external source) o endogenous (i.e., the cytokine has an internal source). In some aspect, the endogenous sources of the cytokine is an ongoing inflammatory condition in a subject, e.g., a chronic condition. In some aspects, the endogenous source of the cytokine is a change in an inflammatory condition, e.g., a flare-up or worsening of some inflammatory condition or an infection. In these two cases, the inflammation would be naturally occurring, i.e., it would not be induced in order to trigger an increase in endogenous cytokines. In other aspects, the inflammation is induced, i.e., there is an external cause that deliberately causes inflammation, which in turn triggers the endogenous release of inflammatory cytokines.

In some aspects, the cytokines used to stimulate the CCL20 promoter are exogenous, for example, they are administered to the subject in order to stimulate the promoter. When the CCL20 promoters of the present disclosure are used for recombinant gene expression, the cytokine used to stimulate the CCL20 promoter can be added to the culture medium.

In some aspects, polynucleotides, compositions, cells, or methods comprising the use CCL20 promoter of the present disclosure (i.e., a CCL20 promoter of any one of SEQ ID NOS:1-5 or a functional fragment or functional variant thereof) can result in improved expression of a heterologous gene with respect to the expression of the same heterologous gene under the control of a reference promoter, e.g., the Serum Amylose 3 (SAA3) promoter, a promoter that can be activated by IL-1 and IL-6. Accordingly, in some aspects, the present disclosure provides methods to improve the expression of a heterologous gene comprising expressing such heterologous gene under the control of a CCL20 promoter of the present disclosure (i.e., a CCL20 promoter of any one of SEQ ID NOS:1-5 or a functional fragment or functional variant thereof), wherein expression under the control of the CCL20 promoter improves expression the heterologous gene by at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 225%, at least about 250%, at least about 275%, at least about 300%, at least about 325%, at least about 350%, at least about 375%, at least about 400%, at least about 425%, at least about 450%, at least about 475%, or at least about 500% with respect to the expression observed using a reference promoter, wherein the reference promoter is an SAA3 promoter.

In some aspects, the present disclosure provides methods to improve the expression of a heterologous gene comprising expressing such heterologous gene under the control of a CCL20 promoter of the present disclosure (i.e., a CCL20 promoter of any one of SEQ ID NOS:1-5 or a functional fragment or functional variant thereof), wherein expression under the control of the CCL20 promoter improves expression the heterologous gene by about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 225%, about 250%, about 275%, about 300%, about 325%, about 350%, about 375%, about 400%, about 425%, about 450%, about 475%, or about 500% with respect to the expression observed using a reference promoter, wherein the reference promoter is an SAA3 promoter.

In some aspects, the present disclosure provides methods to improve the expression of a heterologous gene comprising expressing such heterologous gene under the control of a CCL20 promoter of the present disclosure (i.e., a CCL20 promoter of any one of SEQ ID NOS:1-5 or a functional fragment or functional variant thereof), wherein expression under the control of the CCL20 promoter improves expression the heterologous gene between about 20% and about 30%, between about 30% and about 40%, between about 40% and about 50%, between about 50% and about 60%, between about 60% and about 70%, between about 70% and about 80%, between about 80% and about 90%, between about 90% and about 100%, between about 100% and about 110%, between about 110% and about 120%, between about 120% and about 130%, between about 130% and about 140%, between about 140% and about 150%, between about 150% and about 160%, between about 160% and about 170%, between about 170% and about 180%, between about 180% and about 190%, between about 190% and about 200%, between about 200% and about 225%, between about 225% and about 250%, between about 250% and about 275%, between about 275% and about 300%, between about 300% and about 325%, between about 325% and about 350%, between about 350% and about 375%, between about 375% and about 400%, between about 400% and about 425%, between about 425% and about 450%, between about 450% and about 475%, between about 475% and about 500% with respect to the expression observed using a reference promoter, wherein the reference promoter is an SAA3 promoter.

The present disclosure also provides a gene therapy vector comprising a heterologous gene that upon expression increases or decreases immune activation in a subject, wherein the gene is under the control of a CCL20 promoter of the present disclosure (i.e., a CCL20 promoter of any one of SEQ ID NOS:1-5 or a functional fragment or functional variant thereof). The present disclosure also provides a gene therapy cell comprising a heterologous gene that upon expression increases or decreases immune activation in a subject, wherein the gene is under the control of a CCL20 promoter of the present disclosure (i.e., a CCL20 promoter of any one of SEQ ID NOS:1-5 or a functional fragment or functional variant thereof). The present disclosure also provides personalized medicine method comprising administering a gene therapy vector or cell comprising a heterologous gene that upon expression increases or decreases immune activation in a subject, wherein the gene is under the control of a CCL20 promoter of the present disclosure (i.e., a CCL20 promoter of any one of SEQ ID NOS:1-5 or a functional fragment or functional variant thereof).

The present disclosure also provides diagnostic sensors comprising a CCL20 promoter of the present disclosure (i.e., a CCL20 promoter of any one of SEQ ID NOS:1-5 or a functional fragment or functional variant thereof). As used herein, the term “diagnostic sensor” refers to any compound, material or device, that can be used to indicate the level of inflammation when exposed to an animal body sample comprising inflammatory cytokines capable of stimulating a CCL20 promoter of the present disclosure (i.e., a CCL20 promoter of any one of SEQ ID NOS:1-5 or a functional fragment or functional variant thereof). In some aspects, the diagnostic sensor comprises a CCL20 promoter of the present disclosure (i.e., a CCL20 promoter of any one of SEQ ID NOS:1-5 or a functional fragment or functional variant thereof) operably linker to a reporter gene, e.g., luciferase. In some aspects, the presence of an inflammatory cytokine such IL-1α, IL-1β, TNF-α, TNF-β, IL6, sIL-6R, IL-17, or CD30 in sample elicits the expression of the reporter gene, for example, luciferase, when the diagnostic sensor is contacted with the sampler. Accordingly, the present disclosure provides also methods to determine levels of inflammatory cytokines or levels of inflammation comprising contacting a sample from a subject with a diagnostic sensor disclosed herein.

The present disclosure also provides a tunable regulation system comprising a construct comprising a GAL4-VP16 synthetic transcription factor under control of a CCL20 promoter of the present disclosure (i.e., a CCL20 promoter of any one of SEQ ID NOS:1-5 or a functional fragment or functional variant thereof). GAL4-VP16 promotes transcription of downstream heterologous genes when one or more of its cognate DNA binding sites, UAS (upstream activation sequences), are present. The number of UAS elements can control the amount of transcription of a downstream gene. Thus, the number of UAS elements can be varied to control the level of expression of the heterologous genes located downstream with respect to the UAS elements. In some aspects, the tunable regulation system of the present disclosure comprises a single polynucleotide comprising a CCL20 promoter of the present disclosure (i.e., a CCL20 promoter of any one of SEQ ID NOS:1-5 or a functional fragment or functional variant thereof) operably linked to a GAL4-VP16 synthetic transcription factor, and further downstream in the construct one or more UAS elements operably linked a nucleic acid encoding a heterologous protein. In some aspects, the tunable regulation system of the present disclosure comprises two polynucleotides: (1) a first polynucleotide comprising a CCL20 promoter of the present disclosure (i.e., a CCL20 promoter of any one of SEQ ID NOS:1-5 or a functional fragment or functional variant thereof) operably linked to GAL4-VP16 synthetic transcription factor, and (2) a second polynucleotide comprising one or more UAS sites operably linked to a nucleic acid encoding a heterologous protein.

In some aspects, the present disclosure provides methods to control the expression of a heterologous protein by using a tunable regulation system disclosed herein, wherein the expression level of the heterologous protein correlates with the number of UAS elements in the construct. In some aspects, the tunable regulation system can comprise at least one, at least two, at least three, at least four, at least five, at least six, at least seven, at least eight, at least nine, or at least ten UAS elements. In some aspects, the tunable regulation system can comprise one, two, three, four, five, six, seven, eight, nine, or ten UAS elements.

### III. Indications

The present disclosure provides methods of treating an inflammatory disease or condition is a subject in need thereof comprising administering a construct comprising a CCL20 promoter of the present disclosure (i.e., a CCL20 promoter of any one of SEQ ID NOS:1-5 or a functional fragment or functional variant thereof) operably linked to a heterologous gene to the subject, wherein the stimulation of the CCL20 promoter with a cytokine, e.g., IL-1α, IL-1β, TNF (TNF-α or TNF-β), IL-6, sIL-6R, IL-17, or CD30, induces the expression of the heterologous gene in response to the stimulation. The present disclosure also provides methods of preventing or ameliorating the symptoms of a disease or condition is a subject in need thereof comprising administering a construct comprising a CCL20 promoter of the present disclosure (i.e., a CCL20 promoter of any one of SEQ ID NOS:1-5 or a functional variant thereof) operably linked to a heterologous gene to the subject, wherein the stimulation of the CCL20 promoter with a cytokine, e.g., IL-1α, IL-1β, TNF (TNF-α or TNF-β), IL-6, sIL-6R, IL-17, or CD30, induces the expression of the heterologous gene in response to the stimulation.

The present disclosure also provides methods of preventing and/or treating a disease or disorder in a subject in need thereof, comprising administering a construct comprising a CCL20 promoter of the present disclosure (i.e., a CCL20 promoter of any one of SEQ ID NOS:1-5 or a functional fragment or functional variant thereof) operably linked to a heterologous gene the subject, wherein the stimulation of the CCL20 promoter with a cytokine, e.g., IL-1α, IL-1β, TNF (TNF-α or TNF-β), IL-6, sIL-6R, IL-17, or CD30, induces the expression of the heterologous gene in response to the stimulation. In some aspects, the treatment is prophylactic.

In other aspects, the administering a construct comprising a CCL20 promoter of the present disclosure (i.e., a CCL20 promoter of any one of SEQ ID NOS:1-5 or a functional fragment or functional variant thereof) operably linked to a heterologous gene to the subject, wherein the stimulation of the CCL20 promoter with a cytokine, e.g., IL-1α, IL-1β, TNF (TNF-α or TNF-β), IL-6, sIL-6R, IL-17, or CD30, induces the expression of the heterologous gene in response to the stimulation, can be used to induce an immune response. Thus, in some aspects, a construct comprising a CCL20 promoter of the present disclosure (i.e., a CCL20 promoter of any one of SEQ ID NOS:1-5 or a functional fragment or functional variant thereof) operably linked to a heterologous gene can used to vaccinate a subject.

In some aspects, the compositions and methods disclosed herein can be used to treat inflammatory disorders of the central nervous system (CNS) such as encephalitis, myelitis, meningitis, or arachnoiditis; inflammatory disorders of the peripheral nervous system (PNS) such as neuritis, inflammatory disorders of the eye, dacryoadenitis, scleritis, episcleritis, keratitis, retinitis, chorioretinitis, blepharitis, conjunctivitis, or uveitis; inflammatory disorders of the ear such as otitis externa, otitis media, labyrinthitis, mastoiditis, inflammatory disorders of the cardiovascular system such as carditis, endocarditis, myocarditis, pericarditis, vasculitis, arteritis, phlebitis, or capillaritis; inflammatory disorders of the respiratory system such as sinusitis, rhinitis, pharyngitis, laryngitis, tracheitis, bronchitis, bronchiolitis, pneumonitis, pleuritic, or mediastinitis; inflammatory disorders of the digestive system and accessory digestive organs such as stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, diabetes, proctitis, hepatitis, ascending cholangitis, cholecystitis, or pancreatitis or peritonitis; inflammatory disorders of the integumentary system such as dermatitis, folliculitis, cellulitis or hidradenitis; inflammatory disorders of the musculoskeletal system such as arthritis, dermatomyositis, myositis, synovitis, tenosynovitis, bursitis, enthesitis, fasciitis, capsulitis, epicondylitis, tendinitis, panniculitis, psteochondritis, osteitis, osteomyelitis, spondylitis, seriostitis or chondritis; inflammatory disorders of the urinary system such as nephritis, glomerulonephritis, pyelonephritis, ureteritis, cystitis, or urethritis; inflammatory disorders of the reproductive system such as oophoritis, salpingitis, endometritis, parametritis, cervicitis, vaginitis, vulvitis, mastitis, orchitis, epididymitis, prostatitis, seminal vesiculitis, balanitis, posthitis or balanoposthitis; inflammatory disorders of the endocrine system such as insulitis, hypophysitis, thyroiditis, parathyroiditis, or adrenalitis; or inflammatory disorders of the lymphatic system such as lymphangitis or lymphadenitis.

Inflammatory abnormalities are a large group of disorders that underlie a vast variety of human diseases. The immune system is often involved with inflammatory disorders, as demonstrated in both allergic reactions and some myopathies, with many immune system disorders resulting in abnormal inflammation. In some aspects, the compositions and methods disclosed herein can be used to treat non-immune diseases with causal origins in inflammatory processes, including cancer, atherosclerosis, and ischemic heart disease. Inflammatory abnormalities also include acne vulgaris, rosacea, asthma, atopic eczema, allergic rhinitis, autoimmune diseases (e.g., celiac disease, irritable bowel disease, multiple sclerosis, rheumatoid arthritis, systemic lupus erythematosus, diabetes mellitus type 1, Grave's disease, psoriasis, or aplastic anemia), autoinflammatory diseases (e.g., adult-onset Still's disease, systemic-onset juvenile idiopathic arthritis, Schnitzler syndrome, chronic recurrent multifocal osteomyelitis, Familial Mediterranean fever (FMF), hyperimmunoglobulinemia D with recurrent fever (HIDS), TNF receptor associated periodic syndrome (TRAPS), Muckle-Wells syndrome (urticaria deafness amyloidosis), familial cold urticarial, neonatal onset multisystem inflammatory disease (NOMID), periodic fever, aphthous stomatitis, pharyngitis and adenitis (PFAPA syndrome), Blau syndrome, Pyogenic sterile arthritis, pyoderma gangrenosum, acne (PAPA), deficiency of the interleukin-1-receptor antagonist (DIRA), or Yao syndrome), chronic prostatitis, colitis, diverticulitis, glomerulonephritis, hidradenitis suppurativa, hypersensitivities, inflammatory bowel diseases (e.g., Crohn's disease, ulcerative colitis, Behget's disease, microscopic colitis, diversion colitis, or indeterminate colitis), interstitial cystitis, Lichen planus, mast cell activation syndrome, mastocytosis, otitis, pelvic inflammatory disease, peripheral ulcerative keratitis, pneumonia, reperfusion injury, rheumatic fever, rheumatoid arthritis, rhinitis, sarcoidosis, transplant rejection, vasculitis, atherosclerosis, allergy, anaphylaxis, myopathies (e.g., systemic sclerosis, dermatomyositis, polymyositis, or inclusion body myositis), leukocyte defects (e.g., Chediak-Higashi syndrome or chronic granulomatous disease), pharmacologically-induced inflammation, vitamin-A deficiency, HIV and AIDS, or cancer (e.g., cancers associated with chronic inflammation such as mesothelioma, lung carcinoma, bladder carcinoma, oral squamous cell carcinoma, colorectal carcinoma, vulvar squamous cell carcinoma, pancreatic carcinoma, esophageal carcinoma, salivary gland carcinoma, MALT lymphoma, or melanoma; cancers associated with infectious agents such as cholangiosarcoma, colon carcinoma, gall bladder cancer, gastric adenocarcinoma, hepatocellular carcinoma, B-cell non-Hodgkin's lymphoma, Burkitt;s lymphoma, non-Hodgkin's lymphoma, squamous cell carcinomas, Kaposi's sarcoma, skin carcinoma, ovarian carcinoma, cervical carcinoma, anal carcinoma, bladder carcinoma, liver carcinoma, rectal carcinoma, or follicular lymphoma of the spleen).

In some aspects, inflammation is caused by a pathogen, i.e., a microbe or microorganism such as a virus, bacterium, prior, fungus, or parasite that causes disease in humans. In some aspects, inflammation is caused by a viral disease, e.g., caused by a pathogenic of the families Adenoviridae, Picornaviridae, Herpesviridae, Hepadnaviridae, Coronaviridae, Flaviviridae, Retroviridae, Orthomyxoviridae, Paramyxoviridae, Papovaviridae, Polyomavirus, Poxviridae, Rhabdoviridae, and Togaviridae. Some notable viral diseases include smallpox, influenza, mumps, measles, chickenpox, ebola, rubella, Zika fever, yellow fever, West Nile fever, viral pneumonia, shingles, COVID-19, SARS, MERS, rotavirus infections, Rift Valley fever, respiratory syncytial virus infection, rabies, poliomyelitis, Norovirus infection, Epstein-Barr virus mononucleosis (mono), herpes, hepatitis A, B, C, D or E, Hantavirus pulmonary syndrome, Ebola, Dengue fever, common cold, AIDS, or adenovirus infection. In some aspects, inflammation is caused by a bacterial disease, e.g., anthrax (Bacillus anthracis infection), bacterial meningitis, bacterial pneumonia, bubonic plague (Yersinia pestis infection), botulism (Clostridium botulinum infection), cholera (Vibrio cholerae infection), diphtheria (Corynebacterium diphtheriae infection), epidemic typhus (Rickettsia prowazekii infection), glanders (Burkholderia mallei infection), meningitis (Neisseria meningitidis infection), Pertussis (whooping cough) (Bordetella pertussis infection), salmonellosis, Scarlet fever (Group A Streptococcus species infection), sepsis, shigellosis (bacillary dysentery) (Shigella species infection), Toxic shock syndrome (TSS) (Staphylococcus aureus or Streptococcus pyogenes infection), tuberculosis (Mycobacterium tuberculosis infection), or typhoid fever (Salmonella enterica subsp. enterica, serovar typhi infection).

In some aspects, the disease or disorder that can be treated using a composition or method disclosed herein is a cancer. When a construct comprising a CCL20 promoter of the present disclosure (i.e., a CCL20 promoter of any one of SEQ ID NOS:1-5 or a functional fragment or functional variant thereof) operably linked to a heterologous gene to the subject, wherein the stimulation of the CCL20 promoter with a cytokine, e.g., IL-1α, IL-1β, TNF (TNF-α or TNF-β), IL-6, sIL-6R, IL-17, or CD30, induces the expression of the heterologous gene in response to the stimulation is administered to a subject with a cancer, in certain aspects the expression of the heterologous gene product can up-regulate an immune response and enhance the tumor targeting of the subject's immune system. In other words, whereas in some cases the constructs disclosed herein may be used to decrease inflammation associated with cancer, in other cases the construct disclosed herein can be used to increase inflammation in order to improve tumor targeting. In the first scenario, the presence of endogenous inflammatory cytokine would induce, e.g., the expression of a heterologous gene encoding an anti-inflammatory cytokine. In the second scenario, the presence of low levels of an endogenous inflammatory cytokine many be amplified by a construct of the present disclosure: the stimulation of the CCL20 promoter by endogenous inflammatory cytokines would induce the expression of a pro-inflammatory cytokine.

### IV. Kits and Articles of Manufacture

The present disclosure also provides kits and articles of manufacture comprising the polynucleotides disclosed herein, e.g., a polynucleotide encoding a CCL20 promoter of the present disclosure (i.e., a CCL20 promoter of any one of SEQ ID NOS:1-5 or a functional fragment or functional variant thereof), e.g., a vector.

In some aspects, the present disclosure provides a kit or article of manufacture comprising (i) a CCL20 promoter comprising a sequence set forth in any one of SEQ ID NOS: 1-5 or a functional fragment or functional variant thereof; (ii) a vector comprising a CCL20 promoter comprising a sequence set forth in any one of SEQ ID NOS: 1-5 or a functional fragment or functional variant thereof; or, (iii) a diagnostic construct or sensor comprising a CCL20 promoter comprising a sequence set forth in any one of SEQ ID NOS: 1-5 or a functional fragment or functional variant thereof operably linked to a reporter gene; and instructions for use.

In some aspects, the CCL20 promoter comprising a sequence set forth in any one of SEQ ID NOS: 1-5 or a functional fragment or functional variant thereof is operably linked to a heterologous gene. In some aspects, the vector comprising a CCL20 promoter comprising a sequence set forth in any one of SEQ ID NOS: 1-5 or a functional fragment or functional variant thereof comprises a heterologous gene operably linked to the CCL20 promoter. In some aspects, the diagnostic construct or sensor comprising a CCL20 promoter comprises a sequence set forth in any one of SEQ ID NOS: 1-5 or a functional fragment or functional variant thereof operably linked to a reporter gene comprises a further heterologous gene (e.g., encoding a therapeutic protein or therapeutic nucleotide) also operably linked to the CCL20 (e.g., in a bicistronic construct).

In some aspects, the CCL20 promoter comprising a sequence set forth in any one of SEQ ID NOS: 1-5 or a functional fragment or functional variant thereof is not operably linked to a heterologous gene. In some aspects, the vector comprising a CCL20 promoter comprising a sequence set forth in any one of SEQ ID NOS: 1-5 or a functional fragment or functional variant thereof does not comprise a heterologous gene operably linked to the CCL20 promoter. In some aspects, the diagnostic construct or sensor comprising a CCL20 promoter comprises a sequence set forth in any one of SEQ ID NOS: 1-5 or a functional fragment or functional variant thereof operably linked to a reporter gene, but it does not comprise a further heterologous gene (e.g., encoding a therapeutic protein or therapeutic nucleotide) also operably linked to the CCL20 promoter.

In some aspects, the present disclosure provides a kit or article manufacture comprising (i) polynucleotides disclosed herein, e.g., a polynucleotide encoding a CCL20 promoter of the present disclosure (i.e., a CCL20 promoter of any one of SEQ ID NOS:1-5 or a functional fragment or functional variant thereof), e.g., a vector comprising such promoter), (ii) optionally solvent and/or other reagents, and (iii) instructions explaining, e.g., how to generate a construct comprising a heterologous gene under the control of a CCL20 promoter of the present disclosure.

In some aspects, the present disclosure provides a kit or article manufacture comprising a diagnostic sensor of the present disclosure, and optionally instructions for use. In some aspects, the diagnostic sensor comprises a CCL20 promoter of the present disclosure (i.e., a CCL20 promoter of any one of SEQ ID NOS:1-5 or a functional fragment or functional variant thereof) operably linker to a reporter gene, e.g., luciferase.

In some aspects, the present disclosure provides a kit or article manufacture comprising a tunable expression system of the present disclosure, and optionally instructions for use. In some aspects, the tunable regulation system comprises a single polynucleotide comprising a CCL20 promoter of the present disclosure (i.e., a CCL20 promoter of any one of SEQ ID NOS:1-5 or a functional fragment or functional variant thereof) operably linked to a GAL4-VP16 synthetic transcription factor, and further downstream in the construct one or more UAS elements operably linked a nucleic acid encoding a heterologous protein. In some aspects, the tunable regulation system comprises two polynucleotides: (1) a first polynucleotide comprising a CCL20 promoter of the present disclosure (i.e., a CCL20 promoter of any one of SEQ ID NOS:1-5 or a functional fragment or functional variant thereof) operably linked to GAL4-VP16 synthetic transcription factor, and (2) a second polynucleotide comprising one or more UAS sites operably linked to a nucleic acid encoding a heterologous protein.

Such kits and articles of manufacture can comprise containers, each with one or more of the various components utilized in the methods disclosed herein, including, for example, polynucleotides disclosed herein, e.g., a polynucleotide encoding a CCL20 promoter of the present disclosure (i.e., a CCL20 promoter of any one of SEQ ID NOS:1-5 or a functional fragment or functional variant thereof), e.g., a vector comprising such promoter. In some aspects, the kit comprises printed instructions. Thus, a kit provided according to this disclosure can also comprise brochures or instructions. Instructions included in the kits can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The following examples are offered by way of illustration and not by way of limitation.

### EXAMPLES

### Example 1

### Increased Expression of Genes Under the Control of (Operably Linked to) the CCL20 Promoter in Response to Inflammation

A synthetic construct (designated promoter-less-FF-Luc, or promoterless control), encoding the firefly luciferase gene without any promoter sequence, was also acquired (Promega pGL4.10[luc2]. A synthetic construct (designated CCL20-FF-Luc, or CCL20-Luc), encoding the firefly luciferase gene under the control of (operably linked to) the CCL20 promoter of SEQ ID NO:1, was generated from pGL4.10[luc2]. A third synthetic construct (designated SAA3-FF-Luc), encoding the firefly luciferase under the control of (operably linked to) the SAA3 promoter, was also generated from pGL4.10[luc2]. In addition, a synthetic control construct (designated SV40-FF-Luc), encoding the firefly luciferase under the control of (operably linked to) the SV40 constitutively active promoter, was also acquired (Promega pGL4.35[luc2/SV40].

Four groups of HeLa cells were transfected with Lipofectamine 3000 (Invitrogen) with the CCL20-FF-Luc, the promoter-less-FF-Luc, the SAA3-FF-Luc, or the SV40-FF-Luc constructs and a construct to normalize transfection variance, pGL4.75[hRluc/CMV]. To mimic inflammation conditions the inflammatory cytokine interleukin-1 alpha (IL-1α) was added to each cell culture at a concentration of 5 ng/ml, and luciferase expression (i.e., luciferase reporter assay) was measured using a luciferase reporter assay after 0.5, 2, 4 and 24 hours of IL-1α stimulation. Expression of firefly and Renilla luciferase was determined using the Promega Dual-Glo Luciferase Assay System and read on a plate reader capable of measuring luminescence.

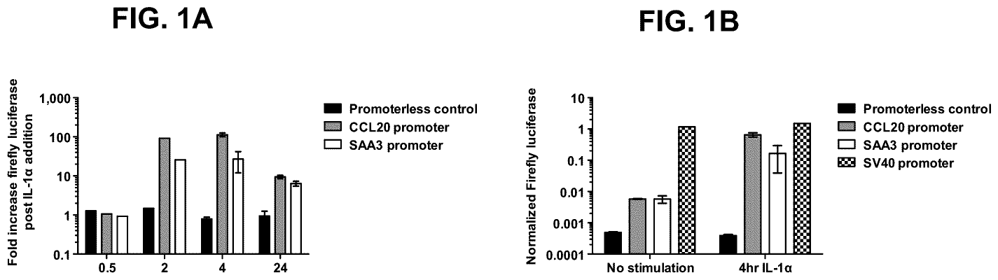

FIG. 1A and FIG. 1B show diagrams describing the fold induction of gene expression, as measured by luciferase expression, induced by each promoter (CCL20, SAA3, or SV40) or promoter-less expression. Luciferase expression levels increased after 2 hours and after 4 hours of IL-1α stimulation, in both CCL20-FF-Luc and SAA3-FF-Luc constructs transfected cells, while luciferase levels remained unchanged at any time in cells transfected with the promoter-less-FF-Luc construct (FIG. 1A).

At 4 hours of IL-1α stimulation, the CCL20 promoter produced 117-fold higher levels of luciferase than in the absence of IL-1α, while the SAA3 promoter produced 27-fold higher levels of luciferase than in the absence of IL-1α. The promoter-less construct produced no difference between the presence or absence of of IL-1α. The SV40 promoter produced high levels of luciferase expression both in the presence or absence of of IL-1α (FIG. 1B). The results of the above luciferase reporter assay, showed that the CCL20 promoter possesses higher efficiency in inducing downstream gene expression in the presence of IL-1α (an agent mimicking inflammation-like condition) compared to the SAA3 promoter. The result also indicate that the CCL20 promoter can effectively induce gene expression in response to stimulation with inflammatory cytokines.

To further investigate the ability of the CCL20 promoter to control specific gene expression levels in response to inflammatory signals (here IL-1α stimulation) a synthetic fusion construct (CCL20-1RA), encoding the Interleukin-1 receptor antagonist (IL-1RA) under the control of (operably linked to) the CCL20 promoter, was generated. A second synthetic construct (CMV-IL-1RA), encoding IL-1RA under the control of the constitutively active CMV promoter, was also generated.

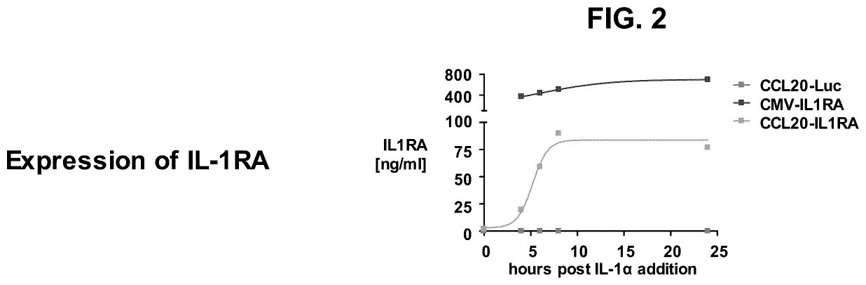

HeLa cells were transfected using Lipofectamine 3000 (Invitrogen) with the CCL20-IL-1RA, CMV-IL-1RA, or CCL20-FF-Luc constructs, and stimulated with 5 ng/ml IL-1α. The levels of IL-1RA expression were measured from 0 hours to 25 hours of IL-1α stimulation. Cells transfected with the CCL20-FF-Luc construct, a control construct not encoding IL-1RA, did not show any IL-1RA expression. Cells transfected with the CMV-IL-1RA (IL-1RA under the control of the constitutively active CMV promoter) showed constant high levels of IL-1RA expression, and showed no changes in IL-1RA expression upon IL-1α stimulation. Cells transfected with the CCL20-IL-1RA construct showed increased IL-1RA expression levels upon IL-1α stimulation (FIG. 2). These results showed that the CCL20 promoter is capable of regulating IL-1RA expression in response to inflammatory signals (here IL-1α).

### Example 2

### Decreased Pro-Inflammatory Cytokine Expression Level Upon Expression of a Pro-Inflammatory Cytokine Under the Control of the CCL20 Promoter and Exposure to Inflammatory Signals

To address if IL-1RA expression under the control of the CCL20 promoter, was capable of reducing pro-inflammatory cytokines expression levels and whether the reduction show any dose-dependency with respect to the amount of inducing cytokine used, HeLa cells were transfected using Lipofectamine 3000 (Invitrogen) with the CCL20-IL-1RA, CMV-IL-1RA, or CCL20-FF-Luc constructs, stimulated with 5 ng/ml IL-1, and the levels of IL-8, IL-6 and MCP-1 in the extracellular environment were measured using Biolegend Legendplex Human Inflammation Panel 1 from 0 hours to 25 hours of IL-1α stimulation.

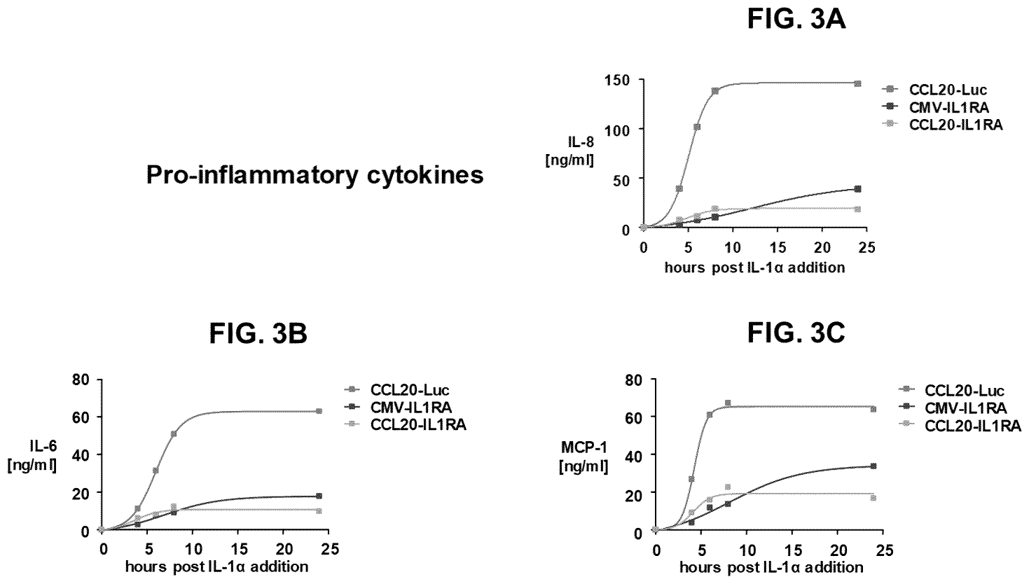

IL-8, IL-6 and MCP-1 expression levels remained high upon stimulation of cells transfected with the CCL20-FF-Luc, a construct not encoding IL-1RA, with IL-1α (FIGS. 3A-C). IL-8, IL-6 and MCP-1 expression levels remained low (did not increase) in response to IL-1α stimulation in cells transfected with the CMV-IL-1RA construct, i.e., cells constitutively expressing high levels of IL-1RA under the control of the constitutively active CMV promoter (FIGS. 3A-C). IL-8, IL-6 and MCP-1 expression levels also remained low (did not increase) in response to IL-1α stimulation in cells transfected with the CCL20-IL-1RA construct, demonstrating the capability of the CCL20 promoter to produce high gene expression levels of IL-1RA upon IL-1α stimulation (FIGS. 3A-C) which can then inhibit downstream inflammation signaling.

These results showed that immune activation can be reduced by expressing a pro-inflammatory cytokine antagonist under the control of the CCL20 promoter. Furthermore, this regulation was at least as equivalent as producing high levels of pro-inflammatory cytokine antagonist present at the time of pro-inflammatory signal initiation (IL-1α stimulation).

### Example 3

### IL-10 Expression in Response to Stimulation of the CCL20 Promoter with IL-1α

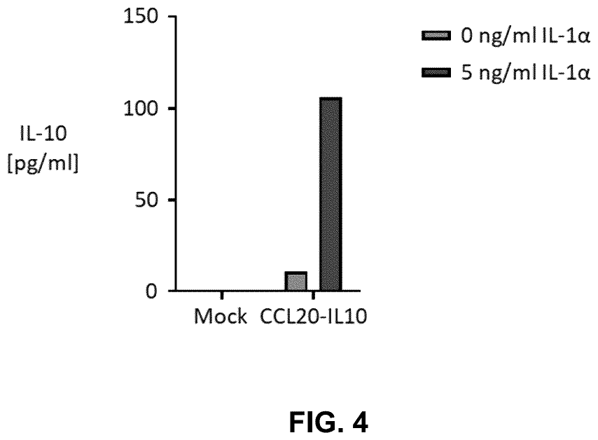

HeLa cells were transfected using Lipofectamine 3000 (Invitrogen) with the CCL20-IL10 constructs, and stimulated with 5 ng/ml IL-1α. The levels of IL-10 expression were measured at 24 hours of IL-1α stimulation. Cells that were not stimulated produced only low levels of the IL-10 immune regulator while stimulation led to high levels of IL-10 stimulation as measured by IL-10 ELISA. See FIG. 4.

### Example 4

### Tunable Second Level Regulation Using the CCL20 Promoter

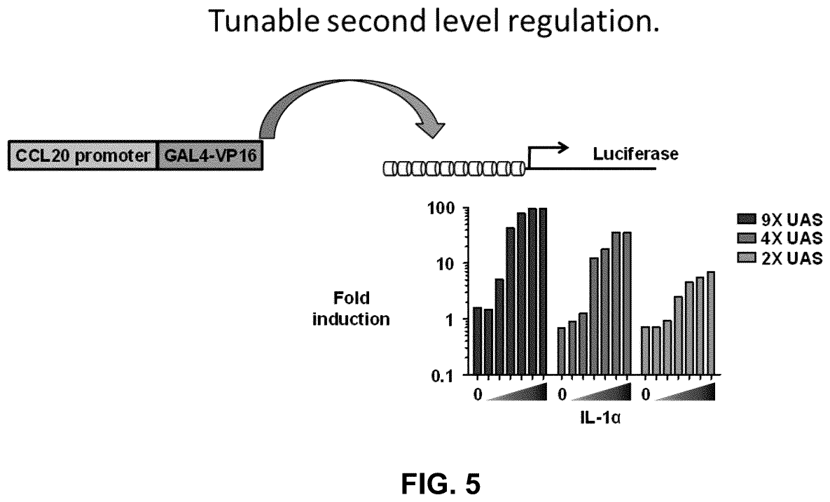

A construct was created with GAL4-VP16, a synthetic transcription factor, under control of the CCL20 promoter. GAL4-VP16 promotes transcription of downstream genes when its cognate DNA binding site, UAS, is present. The number of UAS elements can control the amount of transcription of the downstream gene. A construct that contains 9 UAS sites (Promega pGL4.35[luc2P/9XGAL4UAS/Hygro]) was obtained and used to create a construct with 4 UAS sites and a construct with 2 UAS sites. Each UAS construct was transfected into HeLa cells in concert with the CCL20-GAL4-VP16 construct and the CMV-Renilla luciferase construct. The cells were then stimulated with either 0, 0.05, 0.1, 0.5, 1, 5, or 10 ng/ml IL-1α for 4 hours and luciferase levels were determined by Dual Glo Luciferase Assay system, normalized to Renilla luciferase to account for any transfection variability, then compared to 0 ng/ml stimulation levels to determine the fold increase in luciferase expression. Constructs exhibited a dose dependent response relative to 1) the concentration of IL-1α stimulation and 2) number of UAS elements present in the reporter construct. See FIG. 5.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein.

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

1. A polynucleotide comprising a promoter region comprising a C—C Motif Chemokine Ligand 20 (CCL20) promoter operatively linked to a heterologous gene, wherein the CCL20 promoter induces expression of the heterologous gene in response to stimulation with a cytokine.

2. The polynucleotide of claim 1, wherein the CCL20 promoter comprises the sequence set forth in any one of SEQ ID NOS: 1-5, or a functional fragment or functional variant of the sequence set forth in any one of SEQ ID NOS: 1-5.

3-4. (canceled)

5. The polynucleotide of claim 4, wherein
(i) the functional fragment has a 5′ truncation, a 3′ truncation, or both relative to a CCL20 promoter sequence set forth in any one of SEQ ID NOS: 1-5;
(ii) the functional variant has a sequence with at least about 70%, about 75%, about 80%, about 85%, about 90%, about 95%, about 96%, about 97%, about 98%, or about 99% sequence identity to the sequence set forth in any one of SEQ ID NOS:1-5; or,
(iii) the CCL20 promoter functional variant has 1, 2, 3, 4, 5, 6, 7, 8, 9, 10, 11, 12, 13, 14, 15, 16, 17, 18, 19, or 20 point mutations with respect to sequence set forth in any one of SEQ ID NOS:1-5.

6-8. (canceled)

9. The polynucleotide of claim 1, wherein the heterologous gene encodes a therapeutic protein selected from the group consisting of a chimeric antigenic receptor (CAR), an antibody, a tumor suppressor, an inducer of apoptosis, an enzyme, and a hormone.

10-11. (canceled)

12. A composition comprising (a) a polynucleotide of claim 1, and (b) a delivery vehicle for delivery of the polynucleotide into a cell in a subject.

13. The composition of claim 12, wherein the delivery vehicle is a nucleic acid, a plasmid, a vector, a prokaryotic cell, a eukaryotic cell, or a lipid.

14. The composition of claim 13, wherein
(i) the lipid is comprised in a lipid vesicle selected from the group consisting of a micelle, a liposome, a lipid nanoparticle, and an extracellular vesicle: or
(ii) the vector is a viral vector selected from the group consisting of adeno-associated virus, adenovirus, retrovirus, orthomyxovirus, paramyxovirus, papovavirus, picornavirus, lentivirus, herpes simplex virus, vaccinia virus, pox virus, and alphavirus.

15-16. (canceled)

17. A vector comprising a polynucleotide of claim 1.

18-22. (canceled)

23. A method of producing a cell that expresses at least one heterologous gene comprising introducing into the cell a composition comprising a polynucleotide of claim 1.

24. A cell genetically modified to express a heterologous gene, wherein the cell comprises a polynucleotide, composition, or vector comprising a polynucleotide of claim 1.

25. A pharmaceutical composition comprising (i) a polynucleotide, composition, vector, or cell comprising a polynucleotide of claim 1, and (ii) a pharmaceutically acceptable carrier or excipient.

26. A method of inducing, regulating, or enhancing the expression of a heterologous gene in a subject comprising (a) administering a polynucleotide, composition, vector, cell, or pharmaceutical composition comprising a polynucleotide of claim 1 to the subject; and (b) stimulating the CCL20 promoter with a cytokine.

27. A method of inducing dose-dependent gene expression of a heterologous gene in a cell comprising (a) contacting the cell with a polynucleotide, composition, vector, cell, or pharmaceutical composition comprising a polynucleotide of claim 1; and (b) contacting the cell with an effective amount of a cytokine capable of stimulating the CCL20 promoter to induce expression of the heterologous gene in a dose-dependent manner.

28. (canceled)

29. A method of treating a disease or condition in a subject, comprising administering an effective amount of a polynucleotide, composition, vector, cell, or pharmaceutical composition comprising a polynucleotide of claim 1 to the subject, and optionally stimulating the CCL20 promoter with an exogenous cytokine.

30. (canceled)

31. A method of controlling inflammation in a subject, comprising operatively linking a CCL20 promoter to a heterologous gene, wherein the stimulation of the CCL20 promoter with a cytokine induces the expression of the heterologous gene in response to the stimulation, wherein the heterologous gene controls inflammation by increasing or decreasing immune activation, or increasing or decreasing the expression of a gene that regulates an inflammation pathway.

32. The polynucleotide of claim 1, wherein the cytokine stimulating the CCL20 promoter is selected from the group consisting of IL-1α, IL-1β, TNF-α, IL-6, sIL-6R, IL-17, CD30, or any combination thereof.

33-35. (canceled)

36. The polynucleotide of claim 1, wherein the cytokine stimulating the CCL20 promoter is (i) an exogenous cytokine or (ii) an endogenous cytokine, wherein the endogenous cytokine is the result of naturally occurring or induced inflammation.

37-44. (canceled)

45. A method of producing a recombinant protein comprising culturing a cell comprising a polynucleotide of claim 1, wherein the heterologous gene encodes the recombinant protein.

46. A method of determining the presence of one or more inflammatory cytokines in a sample from a subject comprising (i) contacting the sample with a polynucleotide of claim 1 or cell comprising a polynucleotide of claim 1 wherein the heterologous gene encodes a reporter protein, and (ii) determining the expression of the reporting protein in response to stimulation of the CCL20 promoter by an inflammatory cytokine in the sample.

47. A kit or article of manufacture comprising
(i) a CCL20 promoter comprising a sequence set forth in any one of SEQ ID NOS: 1-5 or a functional fragment or functional variant thereof,
(ii) a vector comprising a CCL20 promoter comprising a sequence set forth in any one of SEQ ID NOS: 1-5 or a functional fragment or functional variant thereof; or,
(iii) a diagnostic construct or sensor comprising a CCL20 promoter comprises a sequence set forth in any one of SEQ ID NOS: 1-5 or a functional fragment or functional variant thereof operably linked to a reporter gene;
and, instructions for use.


SEQUENCE LISTING (5 sequences)

SEQ ID NO: 1 - DNA, 922 bp, synthetic construct
  source 1..922: mol_type = other DNA
  misc_feature 1..922: note = CCL20 promoter full sequence
gagagttcttatactgccttaggttagggattcatattacattatcatcattagtactgttatttgacatttgctgtgctgactagctactgctgataggttttctttcctcaacaattctgaggctctatattgagttatattagtacatcatcatggagagttaaaggtaggtaaggattattttctgaactgcaatattgattaaagccatgtgaatgtataagattcttagaagagttgacattaaatcaaggtgaagctgaggtttgagccttacttaaaggctgatattttccactctaactgcggacagtactgtagcactgttatagtacctgctctgaatgttagtctagcaactcagggtcttcttcatgacagctgaacctcaaccatgtgatggtaaatgtgtagcagagtatgcctggcatcccacctgctcctcctccccctcctccttgactggttctggaaagcaaatagggtgtaacaataggagttctggaatgttcctgtgtggggctgacctttgtatcgctgttaatcctctattttcagacacaaaaatgattaagttaaaactggatgaaagtcttttctgggtcacagggctgagctgcttttgctctttgcaaatacaaagaatttaacaggattctccccttctcaacttcctgtcccccaccctgaccttcgcaccttcccaatatgaggaaaaagcaggaagttttccttgcgggttttttttatgatgacatgatggggccagttgatcaatggggaaaaccccatgtggcaacacgccttctgtgtacattcccaatatttgctataaatagggccatcccaggctgctgtcagaatataacagcactcccaaagaactgggtactcaacactgagcagatctgttctttga

SEQ ID NO: 2 - DNA, 864 bp, synthetic construct
  source 1..864: mol_type = other DNA
  misc_feature 1..864: note = CCL20 promoter without 5' region
gttatttgacatttgctgtgctgactagctactgctgataggttttctttcctcaacaattctgaggctctatattgagttatattagtacatcatcatggagagttaaaggtaggtaaggattattttctgaactgcaatattgattaaagccatgtgaatgtataagattcttagaagagttgacattaaatcaaggtgaagctgaggtttgagccttacttaaaggctgatattttccactctaactgcggacagtactgtagcactgttatagtacctgctctgaatgttagtctagcaactcagggtcttcttcatgacagctgaacctcaaccatgtgatggtaaatgtgtagcagagtatgcctggcatcccacctgctcctcctccccctcctccttgactggttctggaaagcaaatagggtgtaacaataggagttctggaatgttcctgtgtggggctgacctttgtatcgctgttaatcctctattttcagacacaaaaatgattaagttaaaactggatgaaagtcttttctgggtcacagggctgagctgcttttgctctttgcaaatacaaagaatttaacaggattctccccttctcaacttcctgtcccccaccctgaccttcgcaccttcccaatatgaggaaaaagcaggaagttttccttgcgggttttttttatgatgacatgatggggccagttgatcaatggggaaaaccccatgtggcaacacgccttctgtgtacattcccaatatttgctataaatagggccatcccaggctgctgtcagaatataacagcactcccaaagaactgggtactcaacactgagcagatctgttctttga

SEQ ID NO: 3 - DNA, 874 bp, synthetic construct
  source 1..874: mol_type = other DNA
  misc_feature 1..874: note = CCL20 promoter without 3' region
gagagttcttatactgccttaggttagggattcatattacattatcatcattagtactgttatttgacatttgctgtgctgactagctactgctgataggttttctttcctcaacaattctgaggctctatattgagttatattagtacatcatcatggagagttaaaggtaggtaaggattattttctgaactgcaatattgattaaagccatgtgaatgtataagattcttagaagagttgacattaaatcaaggtgaagctgaggtttgagccttacttaaaggctgatattttccactctaactgcggacagtactgtagcactgttatagtacctgctctgaatgttagtctagcaactcagggtcttcttcatgacagctgaacctcaaccatgtgatggtaaatgtgtagcagagtatgcctggcatcccacctgctcctcctccccctcctccttgactggttctggaaagcaaatagggtgtaacaataggagttctggaatgttcctgtgtggggctgacctttgtatcgctgttaatcctctattttcagacacaaaaatgattaagttaaaactggatgaaagtcttttctgggtcacagggctgagctgcttttgctctttgcaaatacaaagaatttaacaggattctccccttctcaacttcctgtcccccaccctgaccttcgcaccttcccaatatgaggaaaaagcaggaagttttccttgcgggttttttttatgatgacatgatggggccagttgatcaatggggaaaaccccatgtggcaacacgccttctgtgtacattcccaatatttgctataaatagggccatcccaggctgctgtcagaatataacag

SEQ ID NO: 4 - DNA, 816 bp, synthetic construct
  source 1..816: mol_type = other DNA
  misc_feature 1..816: note = CCL20 promoter, core region
gttatttgacatttgctgtgctgactagctactgctgataggttttctttcctcaacaattctgaggctctatattgagttatattagtacatcatcatggagagttaaaggtaggtaaggattattttctgaactgcaatattgattaaagccatgtgaatgtataagattcttagaagagttgacattaaatcaaggtgaagctgaggtttgagccttacttaaaggctgatattttccactctaactgcggacagtactgtagcactgttatagtacctgctctgaatgttagtctagcaactcagggtcttcttcatgacagctgaacctcaaccatgtgatggtaaatgtgtagcagagtatgcctggcatcccacctgctcctcctccccctcctccttgactggttctggaaagcaaatagggtgtaacaataggagttctggaatgttcctgtgtggggctgacctttgtatcgctgttaatcctctattttcagacacaaaaatgattaagttaaaactggatgaaagtcttttctgggtcacagggctgagctgcttttgctctttgcaaatacaaagaatttaacaggattctccccttctcaacttcctgtcccccaccctgaccttcgcaccttcccaatatgaggaaaaagcaggaagttttccttgcgggttttttttatgatgacatgatggggccagttgatcaatggggaaaaccccatgtggcaacacgccttctgtgtacattcccaatatttgctataaatagggccatcccaggctgctgtcagaatataacag

SEQ ID NO: 5 - DNA, 192 bp, synthetic construct
  source 1..192: mol_type = other DNA
  misc_feature 1..192: note = CCL promoter, small
cccacctgctcctcctccccctcctccttgactggttctggaaagcaaatagggtgtaacaataggagttctggaatgttcctgtgtggggctgacctttgtatcgctgttaatcctctattttcagacacaaaaatgattaagttaaaactggatgaaagtcttttctgggtcacagggctgagctgcttt